SEQUENCE LISTING (22 sequences)

SEQ ID NO: 1 - DNA, 879 bp, Vigna angularis
atgggttcttctttgtggacttgtctgattctgttatcactggcttctgcttctttcgctgccaacccaagaactccaattgatgtaccatttggcagaaactatgtgcctacttgggcctttgatcatatcaaatatctcaatggaggttctgagattcagcttcatctcgataagtacactggtactggattccagtccaaagggtcatacttgtttggtcacttcagcatgtacataaaattggttcctggtgattcagctggcacagtcactgctttctatttatcgtccacaaacgcagaacatgatgaaatagacttcgagttcttgggaaacagaactgggcaaccatacattttacaaacaaatgtgttcaccggaggcaaaggtgacagagagcagagaatctacctctggtttgaccctacgactcaataccacagatattcagtgctatggaacatgtaccagattgtattctatgtggatgactacccaataagggtgttcaagaacagcaatgacttgggagtgaagttccccttcaatcaaccaatgaaaatatacaacagtttgtggaatgcagatgactgggctacaaggggtggtttggagaaaacagattggtccaaagcccccttcatagcctcttacaagggcttccacattgatgggtgtgaggcctcagtgaatgccaagttctgtgacacacaaggcaagaggtggtgggatcaaccagagtttcgtgaccttgatgctgctcagtggcaaaaactggcttgggtacgcaacaaatacaccatctacaactactgcactgatcgcaaacgctactctcaagtccctccagagtgcaccagagaccgtgacatttaa

SEQ ID NO: 2 - DNA, 876 bp, Vigna angularis
atgggctcgtccctctggacttgtttgatcctcctctccttggcatcggcatccttcgcagcgaaccctcgaactccgatcgatgtgcctttcggacggaactacgtgccgacatgggcattcgaccacattaagtatttgaacggaggctcggagatccagttgcatctcgacaagtacaccggcactggtttccagtcgaagggctcctacttgttcggacatttctccatgtacatcaaattggtgcctggtgactcggcaggaactgtcaccgcattctacctctcgtcgacaaacgcagagcatgacgaaatcgacttcgagttcctcggcaacaggacaggacagccgtacatcctccagaccaacgtcttcacaggaggcaaaggtgatcgggaacagcggatctacttgtggttcgatcccacaacccagtaccataggtactcggtgctctggaacatgtatcagatcgtcttctacgtcgacgattatccgatccgagtgttcaagaactccaacgacttgggcgtcaaattccccttcaaccagcccatgaagatttacaactcgttgtggaacgccgacgattgggcaaccaggggtggtctcgagaagacagattggtcgaaagcacctttcatcgcgtcgtacaagggtttccacatcgacggatgtgaagcctccgtgaacgccaagttctgtgacacccagggcaaacgatggtgggatcagccggaattccgggatttggatgcagcccagtggcagaagctcgcgtgggtcaggaacaagtacaccatctataactactgtaccgatcggaaacgatattcgcaggtgcctcccgagtgtacacgcgatagggacatc

SEQ ID NO: 3 - protein, 292 aa, Vigna angularis
MGSSLWTCLILLSLASASFAANPRTPIDVPFGRNYVPTWAFDHIKYLNGGSEIQLHLDKYTGTGFQSKGSYLFGHFSMYIKLVPGDSAGTVTAFYLSSTNAEHDEIDFEFLGNRTGQPYILQTNVFTGGKGDREQRIYLWFDPTTQYHRYSVLWNMYQIVFYVDDYPIRVFKNSNDLGVKFPFNQPMKIYNSLWNADDWATRGGLEKTDWSKAPFIASYKGFHIDGCEASVNAKFCDTQGKRWWDQPEFRDLDAAQWQKLAWVRNKYTIYNYCTDRKRYSQVPPECTRDRDI

SEQ ID NO: 4 - DNA, 864 bp, Arabidopsis thaliana
atggcgtgtttcgcaaccaaacagcctctgttgttgtctctcctccttgccattggcttctttgtggtggctgcatctgccggaaacttctatgagagctttgatatcacttggggtaatggtcgtgccaacatattcgagaatggacagcttctcacttgtactcttgacaaggtctccggctcaggttttcaatccaagaaggagtacttgtttggtaagatcgacatgaagctcaagcttgtcgctggaaactctgctggcaccgtcaccgcctactacctatcgtcaaaaggcacggcatgggatgagattgacttcgagtttttgggaaatcgcacaggacatccttacactatccacaccaatgtgttcaccggaggtaaaggcgaccgtgagatgcagttccgtctctggttcgatcccactgcggatttccacacctacaccgtccactggaaccctgttaacatcatcttccttgtggatgggatcccaattcgggtgttcaagaacaacgagaaaaatggggtggcttaccctaagaaccagccgatgaggatatactcaagcctttgggaagccgatgactgggctacagaaggcggtcgcgtgaagatcgactggagcaacgcaccattcaaggcctcttacagaaacttcaacgaccaaagctcatgcagcaggacatcaagctcaaaatgggtgacttgcgagccaaacagcaactcgtggatgtggacgactctcaatcctgcccagtacggaaaaatgatgtgggtgcaacgagacttcatgatctacaactattgtaccgattttaagagattccctcaaggcctccccaaggagtgtaaactttga

SEQ ID NO: 5 - DNA, 861 bp, Arabidopsis thaliana
atggcctgtttcgcaaccaaacagccgttgttgctctccttgttgctcgccatcggtttcttcgtggtggcagcctccgcaggaaacttctatgagtccttcgacatcacctggggcaacggaagggcgaacattttcgaaaacggtcagctcctcacttgtacgctcgacaaggtgtccggctccggtttccagtcgaagaaggagtacttgttcggcaagatcgacatgaagctcaagttggtggcaggtaactcggcaggtaccgtcacagcgtactatttgtcgtccaagggaactgcgtgggacgaaatcgacttcgagttcctcggcaaccgtacaggacacccctacactattcacaccaacgtcttcaccggaggcaagggtgatcgggagatgcagttcaggctctggttcgacccgacagcggatttccatacttacacggtgcattggaaccccgtcaacatcattttcctcgtcgacggaatccccatccgagtcttcaagaacaacgagaagaacggtgtggcgtatcccaaaaaccagccgatgcgcatctactcctcgttgtgggaagcggacgactgggccacagaaggcggacgcgtcaagatcgactggtcgaacgcaccgttcaaggcgtcgtaccggaacttcaacgaccagtcgtcctgttcgaggacttcgtcgtccaagtgggtcacctgtgaacccaactcgaactcgtggatgtggactactctcaaccctgcccagtacggcaagatgatgtgggtgcagagggacttcatgatctacaactattgtaccgatttcaaacgattccctcagggtctccccaaggaatgtaaactc

SEQ ID NO: 6 - protein, 287 aa, Arabidopsis thaliana
MACFATKQPLLLSLLLAIGFFVVAASAGNFYESFDITWGNGRANIFENGQLLTCTLDKVSGSGFQSKKEYLFGKIDMKLKLVAGNSAGTVTAYYLSSKGTAWDEIDFEFLGNRTGHPYTIHTNVFTGGKGDREMQFRLWFDPTADFHTYTVHWNPVNIIFLVDGIPIRVFKNNEKNGVAYPKNQPMRIYSSLWEADDWATEGGRVKIDWSNAPFKASYRNFNDQSSCSRTSSSKWVTCEPNSNSWMWTTLNPAQYGKMMWVQRDFMIYNYCTDFKRFPQGLPKECKL

SEQ ID NO: 7 - DNA, 50 bp, Artificial Sequence
  ARTIFICIAL DNA PRIMER
ttcctcaatcctctatatacacaactggccatgggctcgtccctctggac

SEQ ID NO: 8 - DNA, 48 bp, Artificial Sequence
  ARTIFICIAL DNA PRIMER
tgtcagtcacctctagttaattagatgtccctatcgcgtgtacactcg

SEQ ID NO: 9 - DNA, 29 bp, Artificial Sequence
  Artificial DNA Primer
taattaactagaggtgactgacacctggc

SEQ ID NO: 10 - DNA, 31 bp, Artificial Sequence
  Artificial DNA Primer
catggccagttgtgtatatagaggattgagg

SEQ ID NO: 11 - DNA, 36 bp, Artificial Sequence
  Artificial DNA Primer
acatgtctttgataagctagcgggccgcatcatgta

SEQ ID NO: 12 - DNA, 36 bp, Artificial Sequence
  Artificial DNA Primer
tacatgatgcggcccgctagcttatcaaagacatgt

SEQ ID NO: 13 - DNA, 41 bp, Artificial Sequence
  Artificial DNA Primer
ttaatcgccttgcagcacaccgcttcctcgctcactgactc

SEQ ID NO: 14 - DNA, 47 bp, Artificial Sequence
  Artificial DNA Primer
acaataaccctgataaatgcggaacaacactcaaccctatctcggtc

SEQ ID NO: 15 - DNA, 53 bp, Artificial Sequence
  Artificial DNA Primer
agatagggttgagtgttgttccgcatttatcagggttattgtctcatgagcgg

SEQ ID NO: 16 - DNA, 42 bp, Artificial Sequence
  Artificial DNA Primer
ttctacacgaaggaaagaggaggagagagttgaacctggacg

SEQ ID NO: 17 - DNA, 47 bp, Artificial Sequence
  Artificial DNA Primer
aggttcaactctctcctcctctttccttcgtgtagaagaccagacag

SEQ ID NO: 18 - DNA, 43 bp, Artificial Sequence
  Artificial DNA Primer
tcagtgagcgaggaagcggtgtgctgcaaggcgattaagttgg

SEQ ID NO: 19 - DNA, 54 bp, Artificial Sequence
  Artificial DNA Primer
ttcctcaatcctctatatacacaactggccatggcctgtttcgcaaccaaacag

SEQ ID NO: 20 - DNA, 47 bp, Artificial Sequence
  Artificial DNA Primer
agctcgctagagtcgacctagagtttacattccttggggagaccctg

SEQ ID NO: 21 - DNA, 27 bp, Artificial Sequence
  Artificial DNA Primer
taggtcgactctagcgagctcgagatc

SEQ ID NO: 22 - DNA, 40 bp, Artificial Sequence
  Artificial DNA Primer
catggccagttgtgtatatagaggattgaggaaggaagag

(19) United States
(12) Patent Application Publication
(10) Pub. No.: US 2017/0015832 A1
(43) Pub. Date: Jan. 19, 2017

(54) COMPOSITIONS AND PROCESSES FOR IMPROVING PROPERTIES OF FILLERS

(71) Applicant: NOVOZYMES A/S, Bagsvaerd (DK)

(72) Inventors: Alex Berlin, Davis, CA (US); Jason Quinlan, Woodland, CA (US); Romil Benyamino, Sacramento, CA (US); Gregory DeLozier, Raleigh, NC (US)

(73) Assignee: NOVOZYMES A/S, Bagsvaerd (DK)

(21) Appl. No.: 15/123,877

(22) PCT Filed: Mar. 2, 2015
(86) PCT No.: PCT/US2015/018314

§ 371 Date: Sep. 6, 2016

Related U.S. Application Data
(60) Provisional application No. 61/948,240, filed on Mar. 5, 2014.

(51) Int. Cl. C09C 3/10; C09C 1/36; C09D 7/12; D21H 17/69; A61K 8/73; A61Q 19/00; C08B 37/00; C09C 1/42; C12N 9/10

(52) U.S. Cl. CPC C09C 3/10; C09C 1/42; C09C 1/3676; C09D 7/1225; C12N 9/1051; C12Y 204/01207; A61K 8/73; A61Q 19/00; C08B 37/0057; D21H 17/69; A61K 2800/10

(57) ABSTRACT

The present invention relates to processes for modifying a filler material comprising treating the filler material with a composition comprising a xyloglucan endotransglycosylase and (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a a polymeric xyloglucan functionalized with a chemical group; (f) a polymeric xyloglucan; (g) a functionalized xyloglucan oligomer comprising a chemical group; or (h) a xyloglucan oligomer; or a composition of (a-h) without a xyloglucan endotransglycosylase, wherein the modified filler material possesses an improved property compared to the unmodified filler material. The present invention also relates to modified filler materials and modified filler materials obtained by such processes.

### REFERENCE TO A SEQUENCE LISTING

This application contains a Sequence Listing in computer readable form, which is incorporated herein by reference.

### BACKGROUND OF THE INVENTION

Field of the Invention

The present invention relates to compositions and processes for improving properties of filler materials.

Description of the Related Art

Xyloglucan endotransglycosylase (XET) is an enzyme that catalyzes endotransglycosylation of xyloglucan, a structural polysaccharide of plant cell walls. The enzyme is present in most plants, and in particular, land plants. XET has been extracted from dicotyledons and monocotyledons.

Xyloglucan is present in cotton, paper, or wood fibers (Hayashi et al., 1988, Carbohydrate Research 181: 273-277) making strong hydrogen bonds to cellulose (Carpita and Gibeaut, 1993, The Plant Journal 3: 1-30). Adding xyloglucan endotransglycosylase to various cellulosic materials containing xyloglucan alters the xyloglucan mediated interlinkages between the cellulosic fibers improving the strength of the cellulosic materials. WO 97/23683 discloses a process for providing a cellulosic material, such as a fabric or a paper and pulp product, with improved strength and/or shape-retention and/or anti-wrinkling properties by using xyloglucan endotransglycosylase.

Fillers are inert minerals commonly used in a number of products such as paper, cardboard, board, paints, varnishes, lacquers, coatings, beauty and grooming products, building materials, plastics, thermosets, elastomers, rubbers, adhesives, caulkings, asphalt coatings, composites, cements, concrete, sealants, etc. For example, fillers can be introduced to a fiber prior to paper production to reduce the fiber fraction of paper and/or to impart some desirable benefit within the paper, such as strength, barrier, and/or optical properties. In the production of several grades of paper, mineral filler is added to a suspension of a fiber prior to the headbox of the paper machine. A retention aid is typically added to the suspension of the fiber and filler for the purpose of retaining as much filler as possible in the paper. The addition of the filler to the paper imparts several improved properties to the paper sheets such as opacity, whiteness, haptic properties, and printability. Furthermore, the addition of filler to paper can lead to a reduction in the proportion of fiber thereby reducing production costs. Producing paper with higher filler content can result in lower energy costs and increased productivity. The attributes of filler composites prepared from starch and kaolin, in terms of improved retention and altered impact on paper properties, has been documented (Yoon and Deng, 2006, J. Appl. Polym. Sci., 100: 1032-1038).

Fillers are often also added to paints and sealants to reduce cost and impart desired properties to the final product. Filler is most commonly used to replace more costly binder material and thereby reduce cost. Filler is also added to impart color or opacity, and in this capacity the filler is referred to as pigment (e.g., clays, calcium carbonate, mica, silica, talc, titanium dioxide, red iron oxide, etc.). Filler is also added to impart physical properties (e.g., texture, strength, durability, etc.) or to thicken paint. For example, it is known in the art that paints, varnishes or urethane compositions used for high traffic floor applications often contain a high fraction of silica filler to improve the durability of the varnish. Fillers can also be added to glues, where they are commonly referred to as “additives” or “thickeners”.

There is a need in the art to improve the properties of filler materials for use in different industries.

The present invention provides compositions and processes for improving properties of filler materials.

### SUMMARY OF THE INVENTION

The present invention relates to processes for modifying a filler material comprising treating a suspension of the filler material with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase under conditions leading to a modified filler material, wherein the modified filler material possesses an improved property compared to the unmodified filler material.

The present invention also relates to modified filler materials obtained by such processes.

The present invention also relates to modified fillers comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

The present invention also relates to suspensions comprising a filler at least partly coated with a composition comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

The present invention also relates to processes of producing a paper, cardboard, or board, comprising adding such a suspension to a fibrous slurry stock in the production of the paper, cardboard, or board.

The present invention also relates to processes of producing a paint, coating, lacquer, or varnish comprising adding such a suspension to a paint stock, a coating stock, a lacquer stock, or a varnish stock in the production of the paint, coating, lacquer, or varnish.

The present invention also relates to a paper comprising such a modified filler.

The present invention also relates to a cardboard comprising such a modified filler.

The present invention also relates to a board comprising such a modified filler.

The present invention also relates to a paint comprising such a modified filler.

The present invention also relates to a coating comprising such a modified filler.

The present invention also relates to a beauty or grooming product comprising such a modified filler.

The present invention also relates to flocculants for wastewater treatment comprising such a modified filler.

The present invention also relates to building materials comprising such a modified filler.

The present invention further relates to a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase.

### BRIEF DESCRIPTION OF THE FIGURES

FIG. 1 shows a restriction map of pDLHD0012.

FIG. 2 shows a restriction map of pMMar27.

FIG. 3 shows a restriction map of pEvFz1.

FIG. 4 shows a restriction map of pDLHD0006.

FIG. 5 shows a restriction map of pDLHD0039.

FIG. 6 shows the increase of fluorescein isothiocyanate-labeled xyloglucan (FITC-XG) fluorescence associated with the solid phase after incubation with increasing masses of kaolin, relative to a control incubation performed without kaolin. FIG. 6A shows kaolin titration after 1 day of incubation; FIG. 6B shows kaolin titration after 2 days of incubation; and FIG. 7C shows kaolin titration after 5 days of incubation.

FIG. 7 shows fluorescence spectra of supernatants of various kaolin preparations. FIG. 7A shows the fluorescence spectra of supernatants of various kaolin concentrations incubated without FITC-XG. FIG. 7B shows the fluorescence spectra of supernatants of various kaolin concentrations incubated with FITC-XG. FIG. 7C shows the fluorescence spectra of supernatants of various concentrations of kaolin incubated with FITC-XG and Vigna angularis xyloglucan endotransglycosylase 16 (VaXET16).

FIG. 8 shows FITC-XG bound to kaolin by confocal microscopy. FIG. 8A shows the confocal microscopy image of kaolin incubated with no FITC-XG. FIG. 8B shows the confocal microscopy image of kaolin incubated with FITC-XG. FIG. 8C shows the confocal microscopy image of kaolin incubated with FITC-XG and VaXET16. The panels are overlays of transmission and fluorescence emission images.

FIG. 9 shows histograms of pixel intensities for microscope images of kaolin incubated with no FITC-XG, kaolin incubated with FITC-XG, and kaolin incubated with FITC-XG and VaXET16. FIG. 9A shows a pixel intensity histogram for kaolin incubated with no FITC-XG. FIG. 9B shows a pixel intensity histogram for kaolin incubated with FITC-XG. FIG. 9C shows a pixel intensity histogram for kaolin incubated with FITC-XG and VaXET16.

FIG. 10 shows changes in kaolin physical properties after incubation with xyloglucan or xyloglucan and VaXET16. FIG. 10A shows 50 ml conical tubes containing (1) kaolin, (2) kaolin incubated with xyloglucan, and (3) kaolin incubated with xyloglucan and VaXET16 foolowing centrifugation. FIG. 10B shows polystyrene serological pipets following contact with (1) kaolin, (2) kaolin incubated with xyloglucan, and (3) kaolin incubated with xyloglucan and VaXET16. FIG. 10C shows 50 ml conical tubes containing (1) kaolin, (2) kaolin incubated with xyloglucan, and (3) kaolin incubated with xyloglucan and VaXET16 following washing and resuspension in water.

FIG. 11 shows the effects of xyloglucan and VaXET16 modification of kaolin on filler retention in handsheet compositions.

FIG. 12 shows fluorescence intensity of the supernatants of titanium (IV) oxide (TiO₂) binding reactions and control incubations at various times. Open circles: TiO₂ with no FITC-XG; squares: TiO₂ with FITC-XG; diamonds: TiO₂ with FITC-XG and Arabidopsis thaliana xyloglucan endotransglycosylase 14 (AtXET14); triangles: FITC-XG with no TiO₂.

FIG. 13 shows photographs illustrating the changes in TiO₂ physical properties after incubation with xyloglucan or xyloglucan and Arabidopsis thaliana xyloglucan endotransglycosylase 14 (AtXET14).

### DEFINITIONS

As used herein, the singular forms “a”, “an”, and “the” are intended to include the plural forms as well, unless the context clearly indicates otherwise.

Filler material: The term “filler material” means particles added to materials (e.g., plastics, thermosets, elastomers, pulps and papers, rubbers, paints, coatings, varnishes, adhesives, caulkings, asphalt coatings, composites, cements, concretes, sealants, etc.) to reduce their overall end cost, increase their volume, and/or to impart an enhanced property. Examples of filler materials include alumina trihydrate, calcium carbonate (CaCO₃, ground (GCC) or precipitated (PCC)), glass, gypsum (calcium sulfate dehydrate, CaSO₄.2H₂O), kaolin clay (Al₂Si₂O₅(OH)₄, sometimes written as Al₂O₃.2SiO₂.2H₂O), magnesium silicate, mica, silica (silicon dioxide, SiO₂), red iron oxide, titanium dioxide (TiO₂, also called titanium oxide or titanium (IV) oxide), wollastonite (calcium silicate, CaSiO₃), or combinations thereof. In pulp and paper applications, the term “filler material” means material introduced to fiber prior to paper or board production to reduce the fiber fraction of paper or board and/or to impart some desirable benefit within the paper or the board. Pulp and paper fillers are commonly inert minerals and examples include GCC, PCC, kaolin clay, talc (hydrated magnesium silicate, Mg₃Si₄O₁₀(OH)₂, sometimes written as H₂Mg₃(SiO₃)₄), and TiO₂. Filler materials may also be a component of liquid formulations applied as a coat upon the outer surfaces of paper and board to deliver a barrier, strength, and/or optical properties. As optical properties are imparted by both internal and external application of these fillers, they are often referred to as “filler pigments” or “extender pigments”. An extender pigment is used primarily to reduce coating cost, while enhancing coating performance, and is often substituted for more expensive functional color pigments. In plastic, rubber and thermoset applications, filler materials are added to resins to enhance performance and reduce cost. Filler materials may impart enhanced properties such as chemical or corrosion resistance, enhanced impact strength, enhanced shrink-resistance, thermal stability, flame resistance, etc., or may be used to thicken a resin. In paint and coating applications, filler is often used as a lower cost alternative to binder or vehicle components, to impart color or opacity (filler pigments, e.g., clays, calcium carbonate, mica, silica, talc, titanium dioxide, red iron oxide, etc.), to impart physical properties (e.g., texture, strength, durability, etc.), or to thicken a film. In cosmetic applications, the filler may be used as filler pigments designed to impart color or opacity, may be used to impart physical characteristics, or may reduce cost. Mineral cosmetics are composed almost entirely of filler and filler pigment. Waxy or liquid cosmetics contain fillers or filler pigments in addition to oils and waxes that function as binders in the cosmetics.

Functionalized xyloglucan oligomer: The term “functionalized xyloglucan oligomer” means a short chain xyloglucan oligosaccharide, including single or multiple repeating units of xyloglucan, which has been modified by incorporating a chemical group. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The incorporated reactive groups can be derivatized with a compound of interest to directly impart an improved property or to coordinate metal cations and/or to bind other chemical entities that interact (e.g., covalently, hydrophobically, electrostatically, etc.) with the reactive groups. The derivatization can be performed directly on a functionalized xyloglucan oligomer comprising a reactive group or after the functionalized xyloglucan oligomer comprising a reactive group is incorporated into polymeric xyloglucan. Alternatively, the xyloglucan oligomer can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group.

Polymeric xyloglucan: The term “polymeric xyloglucan” means short, intermediate or long chain xyloglucan oligosaccharide or polysaccharide encompassing more than one repeating unit of xyloglucan, e.g., multiple repeating units of xyloglucan. Most optimally, polymeric xyloglucan encompasses xyloglucan of 50-200 kDa number average molecular weight, corresponding to 50-200 repeating units. A repeating motif of xyloglucan is composed of a backbone of four beta-(1-4)-D-glucopyranose residues, three of which have a single alpha-D-xylopyranose residue attached at O-6. Some of the xylose residues are beta-D-galactopyranosylated at O-2, and some of the galactose residues are alpha-L-fucopyranosylated at O-2. The term xyloglucan herein is understood to mean polymeric xyloglucan.

Polymeric xyloglucan functionalized with a chemical group: The term “polymeric xyloglucan functionalized with a chemical group” means a polymeric xyloglucan that has been modified by incorporating a chemical group. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The chemical group can be incorporated into a polymeric xylogucan by reacting the polymeric xyloglucan with a functionalized xyloglucan oligomer in the presence of xyloglucan endotransglycosylase. The incorporated reactive groups can be derivatized with a compound of interest. The derivatization can be performed directly on a functionalized polymeric xyloglucan comprising a reactive group or after a functionalized xyloglucan oligomer comprising a reactive group is incorporated into a polymeric xyloglucan. Alternatively, the polymeric xyloglucan can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group.

Xyloglucan endotransglycosylase: The term “xyloglucan endotransglycosylase” means a xyloglucan:xyloglucan xyloglucanotransferase (EC 2.4.1.207) that catalyzes cleavage of a β-(1→4) bond in the backbone of a xyloglucan and transfers the xyloglucanyl segment on to O-4 of the non-reducing terminal glucose residue of an acceptor, which can be a xyloglucan or an oligosaccharide of xyloglucan. Xyloglucan endotransglycosylases are also known as xyloglucan endotransglycosylase/hydrolases or endo-xyloglucan transferases. Some xylan endotransglycosylases can possess different activities including xyloglucan and mannan endotransglycosylase activities. For example, xylan endotransglycosylase from ripe papaya fruit can use heteroxylans, such as wheat arabinoxylan, birchwood glucuronoxylan, and others as donor molecules. These xylans could potentially play a similar role as xyloglucan while being much cheaper in cost since they can be extracted, for example, from pulp mill spent liquors and/or future biomass biorefineries.

Xyloglucan endotransglycosylase activity can be assayed by those skilled in the art in any of the following methods. Reduction of average molecular weight of the xyloglucan polymer by incubation of xyloglucan with a molar excess of xyloglucan oligomer in the presence of xyloglucan endotransglycosylase can be determined via liquid chromatography (Sulova et al., 2003, Plant Physiol. Biochem. 41: 431-437) or via ethanol precipitation (Yaanaka et al., 2000, Food Hydrocolloids 14: 125-128) followed by gravimetric or cellulose-binding analysis (Fry et al., 1992, Biochem. J. 282: 821-828), or can be assessed colorimetrically by association with iodine under alkaline conditions (Sulova et al., 1995, Analytical Biochemistry 229: 80-85). Incorporation of a functionalized xyloglucan oligomer into xyloglucan polymer by incubation of the functionalized oligomer with xyloglucan in the presence of xyloglucan endotransglycosylase can be assessed, e.g., by incubating a radiolabeled xyloglucan oligomer with xyloglucan and xyloglucan endotransglycosylase, followed by filter paper-binding and measurement of filter paper radioactivity, or incorporation of a fluorescently or optically functionalized xyloglucan oligomer can be assessed similarly, monitoring fluorescence or colorimetrically analyzing the filter paper.

Xyloglucan oligomer: The term “xyloglucan oligomer” means a short chain xyloglucan oligosaccharide, including single or multiple repeating units of xyloglucan. Most optimally, the xyloglucan oligomer will be 1 to 3 kDa in molecular weight, corresponding to 1 to 3 repeating xyloglucan units.

### DETAILED DESCRIPTION OF THE INVENTION

The present invention relates to processes for modifying a filler material comprising treating a suspension of the filler material with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase, under conditions leading to a modified filler material, wherein the modified filler material possesses an improved property compared to the unmodified filler material.

A suspension of the filler material can be any mixture. In one aspect, the suspension is a slurry. In another aspect, the suspension is an aqueous slurry. In another aspect, the suspension is a non-aqueous slurry. In another aspect, the suspension is a partially aqueous slurry. In another aspect, the suspension is a waxy suspension. In another aspect, the suspension is an emulsion. In another aspect the suspension is a gel or hydrogel.

The present invention also relates to modified filler materials obtained by such processes.

The present invention also relates to modified filler materials comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer,

The present invention also relates to suspensions comprising a filler at least partly coated with a composition comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group, and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

The present invention also relates to processes of producing a paper, cardboard, or board, comprising adding such a suspension to a fibrous slurry stock in the production of the paper, cardboard, or board.

The present invention also relates to processes of producing a paint, coating, lacquer, or varnish comprising adding such a suspension to a paint stock, a coating stock, a lacquer stock, or a varnish stock in the production of the paint, coating, lacquer, or varnish.

The present invention also relates to a paper comprising such a modified filler.

The present invention also relates to a cardboard comprising such a modified filler.

The present invention also relates to a board comprising such a modified filler.

The present invention also relates to a paint comprising such a modified filler.

The present invention also relates to a coating comprising such a modified filler.

The present invention also relates to a beauty or grooming product comprising such a modified filler.

The present invention also relates to flocculants for wastewater treatment comprising such a modified filler.

The present invention also relates to building materials comprising such a modified filler.

The present invention further relates to a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase.

In one embodiment, the composition comprises a xyloglucan endotransglycosylase, a polymeric xyloglucan, and a functionalized xyloglucan oligomer comprising a chemical group. In another embodiment, the composition comprises a xyloglucan endotransglycosylase, a polymeric xyloglucan functionalized with a chemical group, and a functionalized xyloglucan oligomer comprising a chemical group. In another embodiment, the composition comprises a xyloglucan endotransglycosylase, a polymeric xyloglucan functionalized with a chemical group, and a xyloglucan oligomer. In another embodiment, the composition comprises a xyloglucan endotransglycosylase, a polymeric xyloglucan, and a xyloglucan oligomer. In another embodiment, the composition comprises a xyloglucan endotransglycosylase and a polymeric xyloglucan functionalized with a chemical group. In another embodiment, the composition comprises a xyloglucan endotransglycosylase and a polymeric xyloglucan. In another embodiment, the composition comprises a xyloglucan endotransglycosylase and a functionalized xyloglucan oligomer comprising a chemical group. In another embodiment, the composition comprises a xyloglucan endotransglycosylase and a xyloglucan oligomer. In each of the embodiments above, the composition comprises no xyloglucan endotransglycosylase.

The processes of the present invention provide modified filler materials that are at least partly coated with a polymeric xyloglucan, a polymeric xyloglucan functionalized with a chemical group, a xyloglucan oligomer, and/or a functionalized xyloglucan oligomer comprising a chemical group, and processes for their preparation and their use. The modified filler materials can be prepared by mixing a suspension of a filler material with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase and then recovering the modified filler materials for use as an additive.

In one aspect, the functionalization can provide any functionally useful chemical moiety.

The xyloglucan endotransglycosylase is preferably present at about 0.1 nM to about 1 mM, e.g., about 10 nM to about 100 μM or about 0.5 to about 5 μM, in the composition.

The polymeric xyloglucan or polymeric xyloglucan functionalized with a chemical group is preferably present at about 1 mg to about 1 g per g of the composition, e.g., about 10 mg to about 9500 mg or about 100 mg to about 900 mg per g of the composition.

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 1 mg to about 1 g per g of the composition, e.g., about 10 mg to about 950 mg or about 100 mg to about 900 mg per g of the composition.

When present with polymeric xyloglucan or polymeric xyloglucan functionalized with a chemical group, the xyloglucan oligomer or the functionalized xyloglucan oligomer is preferably present with the polymeric xyloglucan or polymeric xyloglucan functionalized with a chemical group at about 50:1 to about 0.5:1 molar ratio of xyloglucan oligomer or functionalized xyloglucan oligomer to polymeric xyloglucan or polymeric xyloglucan functionalized with a chemical group, e.g., about 10:1 to about 1:1 or about 5:1 to about 1:1 molar ratio of xyloglucan oligomer or functionalized xyloglucan oligomer to polymeric xyloglucan or polymeric xyloglucan functionalized with a chemical group.

The polymeric xyloglucan or polymeric xyloglucan functionalized with a chemical group is preferably present at about 1 mg to about 1 g per g of the filler material, e.g., about 10 mg to about 100 mg or about 20 mg to about 50 mg per g of the filler material.

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 1 mg per g to about 1 g per g of the filler material, e.g., about 10 mg to about 100 mg or about 20 mg to about 50 mg per g of the filler material.

When present with polymeric xyloglucan or polymeric xyloglucan functionalized with a chemical group, the xyloglucan oligomer or the functionalized xyloglucan oligomer is preferably present with the polymeric xyloglucan or polymeric xyloglucan functionalized with a chemical group at about 50:1 to about 0.5:1 molar ratio of xyloglucan oligomer or functionalized xyloglucan oligomer to polymeric xyloglucan or polymeric xyloglucan functionalized with a chemical group, e.g., about 10:1 to about 1:1 or about 5:1 to about 1:1 molar ratio of xyloglucan oligomer or functionalized xyloglucan oligomer to polymeric xyloglucan or polymeric xyloglucan functionalized with a chemical group.

The xyloglucan endotransglycosylase is preferably present at about 0.1 nM to about 1 mM, e.g., about 10 nM to about 100 μM or about 0.5 to about 5 μM.

The modified filler materials may be used as a component in a number of products. Non-limiting examples include paper, cardboard, board, paints, varnishes and laquers, coatings, beauty and grooming products, building materials, plastics, thermosets, elastomers, pulp and paper, rubber, adhesives, caulkings, asphalt coatings, composites, cements, concrete, and sealants.

For example, the modified filler materials can be used in the production of paper and boards having high filler content. Mineral fillers are commonly introduced as aqueous slurries (15-50% solids) into the fiber stock within chests or pumps in the latter stages of the stock preparation prior to the headbox of the paper or board machine. The fillers are intrinsically inert and have limited attraction to or even repulsion from cellulosic fiber. Therefore, to ensure that filler particles are effectively retained within the embryonic web of fiber during consolidation with a paper or board machine, polymeric additives (e.g., polyacrylamide, polyethyleneimine, poly-(aminoamide)-epichlorohydrin, etc.) are customarily blended with the fiber stock at a point downstream of filler addition. The primary mechanisms of filler retention by such polymeric “retention aids” includes physical entrapment and/or anchoring. The presence of polymeric xyloglucan and/or xyloglucan oligomer, functionalized or nonfunctionalized, permits a surprising degree of incorporation of the filler into the cellulosic paper and board-making fibers, enabling target levels of retention in the absence of or with reduced quantities of retention aid. In addition to a potential cost advantage, the reduction/avoidance of polymeric additives may improve product quality by reducing the potential detriments of polymer imbalances (e.g., deposits, poor formation, over-charging/charge reversal, etc.).

The modified filler materials can be generated separately from the process of manufacturing the final product (e.g., paper, board, coatings, paints, adhesives, cosmetics, and other items), or during the manufacturing process. Mineral fillers (e.g., kaolin, TiO₂, silica, aluminum oxides or aluminum hydrates) are incubated in the presence of xyloglucan endotransglycosylase with (a) a polymeric xyloglucan, and a functionalized xyloglucan oligomer comprising a chemical group, (b) a polymeric xyloglucan functionalized with a chemical group and a functionalized xyloglucan oligomer comprising a chemical group, (c) a polymeric xyloglucan functionalized with a chemical group and a xyloglucan oligomer, (d) a polymeric xyloglucan and a xyloglucan oligomer, (e) a polymeric xyloglucan functionalized with a chemical group, (f) a polymeric xyloglucan, (g) a functionalized xyloglucan oligomer comprising a chemical group, or (h) a xyloglucan oligomer, under conditions leading to the modification or coating of mineral fillers. The incubation is performed for suitable times at suitable temperatures and under suitable reaction conditions to effect a modification. In some aspects, xyloglucan endotransglycosylase can be excluded from the incubation.

When performed separately from a manufacturing process, the process can be performed in batch or in continuous reactors. The modified filler material is recovered by centrifugation, filtration, drying or by settling and removal of excess liquid phase. The xyloglucan endotransglycosylase, unbound polymeric xyloglucan, functionalized or nonfunctionalized, and/or unbound xyloglucan oligomer, functionalized or nonfunctionalized, may be removed by washing (e.g., by repeated dilution and settling, by flowthrough with buffer or water or by any other means known in the art). In some aspects, the polymeric xyloglucan, xyloglucan oligomer, and xyloglucan endotransglycosylase are not separated and the modified filler material is utilized with these components present (i.e., in crude suspension). The modified filler material can be dried or retained in slurry.

When modified filler material is generated during or concurrent with the manufacturing process, filler material is incubated as a suspension in an agitator at a suitable dry weight percentage for the process with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, under suitable conditions to effect a modification of the filler. The agitated slurry is mixed for sufficient time and suitable temperature to effect modification, then the filler slurry is added to the blend chest, machine chest or stuff box as appropriate for the process. In one aspect, the unbound components are removed by washing (e.g., repeated dilution and separation of the liquid phase) in the agitator. In another aspect, the unreacted components are not removed. In another aspect the extent of modification is optimized by addition of the components of one of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, at different stages of the production process to optimize the degree of modification by optimizing the relative times that the components are incubated together. In another aspect, the mineral filler and (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, are incubated under conditions suitable to effect a modification, followed by alteration of the conditions (e.g., adjustment of temperature to less than 5 or greater than 75° C., adjustment of pH to less than 4 or greater than 9, addition of denaturant, and/or addition of organic solvent, etc.) to prevent further modification. This is then followed by addition of the modified filler material or the unseparated reaction mixture to the paper or board making process.

The modification of filler materials according to the present invention provides one or more benefits in other manufacturing processes or products beyond the paper and board industry. While not inclusive, a selection of industrial segment beneficiaries includes paints, coatings, sealants, and finishes, i.e., paints, coatings, lacquers, or varnishes (e.g., rheology modification, functionalization), beauty or grooming products (e.g., cosmetics, toothpaste), flocculants (e.g. wastewater treatment), and building materials (e.g., caulking, ceramic, roofing, rubber, and sealants).

The modified filler materials can also be used in the production of paints, coatings, sealants, and finishes. In the process of paint and coating manufacture, dry powder fillers, along with pigments, filler pigments, other additives, etc. are typically mixed with a small amount of resin and solvent to form a paste. The paste is then dispersed in one of two ways; either in a sand mill, wherein the pigment is ground and dispersed by agitation of silica or sand, or in a high speed (rotary) dispersion tank. Pastes dispersed by means of a sand mill must subsequently be filtered to remove the silica. The pastes are then diluted into appropriate volumes of the desired type of solvent, mixed thoroughly, and packaged or canned for use. The use of modified filler materials can assist the dispersion process, permitting better blending and reducing the energy and time required. Recent California law regarding volatile organic compounds requires that solvent be present at no higher than 250 g/L of paint. A larger fraction of paint formulations must therefore be filler, pigment or other solids, and there is need in the art for enhanced filler compositions. In the processes of the present invention, modified filler materials would be generated separately from or during the paints or coatings manufacture. In one aspect, modified filler materials are generated separately and are used in place of conventional dry powder fillers and pigments. In another aspect, modified filler materials are generated, by incubation of a filler material with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, under suitable conditions to effect a modification of the filler during the dispersion process. This is a preferred aspect when the solvent for the paint is water or aqueous solution (e.g., latex paint, also known as emulsion paint). In another aspect, the modified filler materials are generated during the dilution of the paste in a solvent, particularly in the case of a water-based solvent.

The modified filler materials can also be used in the production of beauty and grooming products. Mineral cosmetics refer to those cosmetics formulated as loose powders, particularly foundation, blush, etc., consisting almost entirely of filler pigments. Examples of these fillers include TiO₂, and oxides of zinc, iron or tin. The fillers are blended in rotary blenders, and compressed into tablets or wafers for packaging into compacts, for instance. Liquid and waxy cosmetics (e.g., lipstick) are typically manufactured by blending filler pigments (e.g., TiO₂, silica, etc.) with oils (e.g., mineral oil, cocoa oil, silicon oil, petrolatum, castor oil, etc.) to generate a paste. Colors are blended by dispersion and grinding, often using a roller mill. Waxes such as candelilla wax, paraffin or carnauba are melted at elevated temperature and mixed with the filler pigment paste in a rotary blender. The formulation is poured into molds and cooled before packaging at low temperature. As mineral cosmetics are almost entirely filler materials, modified filler materials can be used to impart color to the cosmetic, to allow better compression of the cosmetic, to impart improved physical characteristics such as resistance to cracking or breaking, or to improve blending. In the processes of the present invention, the filler can be modified prior to manufacture of the mineral, liquid or waxy cosmetic and utilized as a dry powder or slurry, or it can be modified during manufacture of the cosmetic.

In one aspect, the filler is incubated with (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer, separately from the other pigment fillers, modified and then dried, prior to blending with the other fillers. In another aspect, one or more fillers and filler pigments are mixed together prior to blending with (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer. In another aspect, modified filler material is generated following blending of the mineral cosmetic, but prior to compression into the compact tablet, and the filler mixture is either dried or is left as a slurry, thereby imparting greater capacity for compression or better physical properties of the final product. In another aspect, the generation of modified filler, particularly for liquid or waxy cosmetics, can be performed in the filler paste, prior to addition of hot wax, or can be performed during blending of the hot wax with the paste. In this aspect, it is preferable to utilize a xyloglucan endotransglycosylase with high melting temperature or thermotolerance.

The modified filler materials can also be used in wastewater treatment. Clay minerals are commonly used in flocculant mixtures to help remove suspended solids, fats, and heavy metals. Clays modified with quatenary amines are used to remove mechanically emulsified oil and grease as well as other soluble organics. Also, 10% clay/90% sand mixtures fortified with pebbles have proven effective and suggested as natural water filters for developing countries. Thus, clay minerals can be modified by functionalized xyloglucan to capture a wider variety of pollutants in wastewater.

The modified filler materials can also be used in the production of building materials. As an example, polyvinylchloride (PVC) is often used in building materials and as a wood replacement. TiO₂ filler is commonly utilized in the process of manufacturing PVC building materials. In an additional example, wood plastic composites (WPC) are a relatively new building material commonly used in decks, window and door componentsand fencing. These are approximately 50:50 mixtures of finely ground wood or cellulosic materials (i.e. wood flour) and thermoset plastics (e.g., polystyrene, polyvinylchloride, polyethylene, etc.). Advantages to the composites include reduced environmental impact, lower cost, and greater stiffness than can be achieved with plastics alone. Disadvantages include a tendency to fade in color due to sunlight, thus there is need in the art to prevent UV-damage. To generate the WPC, plastics are melted at temperatures less than 220° C. and are blended or dispersed with wood flour in a compounder or blender along with lubricants and coupling agents designed to enhance the association between the synthetic polymer and the wood flour. Fillers such as talc, filler pigments (referred to as colorants) and additives such as biocidal compounds, UV protectants, or flame retardants may be added at this stage. The blended material is then formed into a desired shape (i.e., boards), embossed with a grain pattern and cut to the correct length. In the processes of the present invention, modified filler materials can be generated during WPC manufacture, or separately from the WPC manufacture. In one aspect, modified filler pigment can be used. In another aspect, modified filler materials other than pigment (e.g., talc, TiO₂, silica, etc.) can be used. The use of modified filler materials may increase the association between cellulose fibers of the wood flour and the plastic resin, thereby reducing the need for coupling agents, reducing the overall cost, and/or improving one or more properties of the WPC. Filler materials may be modified with xyloglucan or xyloglucan oligomers functionalized with UV-resistant properties (e.g., TiO₂, AlO₂, ZnO₂), reducing the need for some additives. Modified filler materials may enhance the association between plastic and cellulose, allowing alteration of the ratios of plastic or wood flour while maintaining the physical properties of the WPC. As water must be excluded from WPC blends during manufacture, in the processes of the present invention, filler and additives are incubated with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, under suitable conditions to effect a modification of the filler, and then dried prior to addition to the compounder. In the processes of the present invention, alternatively, a slurry of the wood flour and the fillers can be incubated with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, under suitable conditions to effect a modification of the filler, then dried prior to blending. In one aspect, unreacted components are separated from the modified filler or the wood flour/modified filler mixture prior to use. In another aspect, the unreacted components are not separated. In the processes of the present invention, the WPC material may be subsequently brought into contact with xyloglucan and xyloglucan endotransglycosylase in an aqueous medium for functionalization after synthesis.

### Filler Materials

In the processes of the present invention, the filler material can be any filler material.

The filler material may be a material composed of alumina, calcium carbonate, calcium sulfate, calcium silicate, glass, kaolin clay, magnesium silicate, mica, red iron oxide, silicon dioxide, titanium dioxide, or combinations thereof as non-limiting examples. The present invention encompasses the modification of all sub-classes of each of the common filler types used within the paper and board industry and includes hydrous, calcined and/or delaminated kaolin; rutile and anatase titanium dioxide; natural (i.e., limestone) or precipitated (scalenohedral, prismatic, rhombohedral and acicular) calcium carbonate, and talc. The filler materials typically have a particle size range of 0.1 to 10 μm.

### Improved Properties

Treatment of a filler material according to the processes of the present invention imparts an improved property to the modified filler materials.

For paper, cardboard, and other paper/cardboard products, the improved property, at a constant filler content, is one or more improvements including, but are not limited to,an increase in dry paper strength, an increase in paper density, a decrease in paper sheet thickness, a modification of paper stiffness, an increase in tear strength, improved opacity, improved brightness, improved printability, and reduced dusting/linting.

For paints, coatings, sealants, and finishes, the improved property, at a constant filler content, is one or more improvements including, but are not limited to,improved paint or coating thickness, fluidity, adhesion to surface, resistance to flaking, cracking or peeling, strength and durability, improved color or appearance, improved resistance to color fading, improved resistance to adverse waether conditions (e.g., sun damage), improved package stability, improved application characteristics, and corrosion resistance.

For beauty or grooming products, the improved property, at a constant filler content, is one or more improvements including, but are not limited to,improved compressibility, improved fluidity, improved opacity, improved color, improved texture, improved adhesion, reduced allergenicity, reduced skin sensitivity, reduced comedogenicity, improved resistance to cracking or breaking, enhanced UV protection, enhanced anti-microbial properties, enhanced stability, resistance to phase-separation, and improved viscosity.

For wastewater treatment, the improved property, at a constant filler content, is one or more improvements including, but are not limited to, enhanced adsorption and flocculant properties for better removal of various water pollutants.

For building materials, the improved property, at a constant filler content, is one or more improvements including, but are not limited to,enhanced mechanical or physical properties, enhanced UV-protection, enhanced flexibility, enhanced opacity, enhanced color, enhanced resistance to color fading, and enhanced resistance to flame or flame retardance.

The modified filler materials improve each of the properties above at a specific content relative to product containing the same content of unmodified filler.

An increase in dry paper strength means a significant improvement in the tensile, burst, and tear strength indices as determined by standard methods described in Tappi Test Methods T494, T414 and T403/807, respectively, or comparable methods.

A decrease in paper sheet thickness means a significant decrease in caliper as measured according to Tappi Standard T411/551 or comparable test method

A modification of paper stiffness means a significant change in the bending stiffness of the sheet as determined according to Tappi standard T556/566 or comparable test method.

An improvement in thickness can be determined by ASTM D7489-09, D1005, or D1212, or before polymerization by ASTM D6606.

An improvement in fluidity can be determined by ASTM D4212-10.

An improvement in adhesion to surfaces can be determined by ASTM D4541. D5179, D2197, or D3359.

An improvement in resistance to flaking, cracking, checking, blistering, or chalking can be assessed by ASTM D2486-06, D660, D661, D662, D714, D772, D1654, or D4214.

An improvement in color or appearance can be determined by ASTM D3928-00a, D5326-94a, D2244, D1360, D332, or D344.

An improvement in resistance to color fading can be assessed by ASTM D1729 or D2616.

An improvement in resistance to sun damage or sun fading can be determined by ASTM D5894.

An improvement in application characteristics can be determined using ASTM D4400-99, D4707-09, D4958-10, or D7073-05.

An improvement in anti-microbial characteristics can be determined using ASTM D2574-06, D3273-12, D3274, or D5590.

An improvement in flame retardancy can be determined using ASTM D1360.

An improvement in water repellency can be assessed using ASTM D5401-03 or D4446-08.

An improvement in package stability can be assessed with ASTM D1849-95.

An improvement in chemical resistance of mortars, grouts, surfacings and polymer concretes can be assessed using ASTM C267.

An improvement in durability can be determined by ASTM D2370, D2134, D3363, or D4060.

The improvement in anti-microbial properties can be determined according to ISO 11930:2012, USP 61, USP 51, preservative challenge test, etc.

The improvement in stability can be determined according to ISO/AWI TR 18811.

An improvement in resistance to changes in texture, viscosity, color, pH, phase-separation, etc., can be determined by accelerated shelf life testing or accelerated physical stability testing.

The improvement in UV-protection can be determined according to ISO 24443:2012, ISO 24444:2010, or ISO 24443:2012

An improvement in skin sensitivity, allergenicity and comedogenicity can be determined according to in vitro dermal irritancy, ocular irritancy and dermal sensitization testing.

The improvement in mechanical or physical properties of WPCs can be determined according to ASTM D 7031-04, Guide for Evaluating Mechanical and Physical Properties of Wood-plastic Composite Products, ASTM D 7032-04, Specification for Establishing Performance Ratings for Wood-plastic Composite Deck Boards and Guardrail Systems Guards or Handrails, ASTM D 6662-01, Specification for Polyolefin-based Plastic Lumber Decking Boards.

The improvement in flame resistance of WPCs and building materials can be determined according to standards 12-7A-1, 12-7A-2 or 12-7A-5, Fire resistive standards for exterior wall siding and sheathing, windows, and decks or other horizontal structures, respectively.

### Polymeric Xyloglucan

The polymeric xyloglucan can be any xyloglucan. In one aspect, the polymeric xyloglucan is obtained from natural sources. In another aspect, the polymeric xyloglucan is synthesized from component carbohydrates, UDP- or GDP-carbohydrates, or halogenated carbohydrates by any means used by those skilled in the art. In another aspect, the natural source of polymeric xyloglucan is tamarind seed or tamarind kernel powder, nasturtium, or plants of the genus Tropaeolum particularly Tropaeolum majus. The natural source of polymeric xyloglucan may be seeds of various dicotyledonous plants such as Hymenaea courbaril, Leguminosae-Caesalpinioideae including the genera Cynometreae, Amherstieae, and Sclerolobieae. The natural source of polymeric xyloglucan may also be the seeds of plants of the families Primulales, Annonaceae, Limnanthaceae, Melianthaceae, Pedaliaceae, and Tropaeolaceae or subfamily Thunbergioideae. The natural source of polymeric xyloglucan may also be the seeds of plants of the families Balsaminaceae, Acanthaceae, Linaceae, Ranunculaceae, Sapindaceae, and Sapotaceae or non-endospermic members of family Leguminosae subfamily Faboideae. In another aspect, the natural source of polymeric xyloglucan is primary cell walls of dicotyledonous plants. In another aspect, the natural source of polymeric xyloglucan may be primary cell walls of nongraminaceous, monocotyledonous plants.

The natural source polymeric xyloglucan may be extracted by extensive boiling or hot water extraction, or by other processes known to those skilled in the art. In one aspect, the polymeric xyloglucan may be subsequently purified, for example, by precipitation in 80% ethanol. In another aspect, the polymeric xyloglucan is a crude or enriched preparation, for example, tamarind kernel powder. In another aspect, the synthetic xyloglucan may be generated by automated carbohydrate synthesis (Seeberger, Chem. Commun, 2003, 1115-1121), or by means of enzymatic polymerization, for example, using a glycosynthase (Spaduit et al., 2011, J. Am. Chem. Soc. 133:10892-10900).

In one aspect, the average molecular weight of the polymeric xyloglucan ranges from about 2 kDa to about 500 kDa, e.g., about 2 kDa to about 400 kDa, about 3 kDa to about 300 kDa, about 3 kDa to about 200 kDa, about 5 kDa to about 100 kDa, about 5 kDa to about 75 kDa, about 7.5 kDa to about 50 kDa, or about 10 kDa to about 30 kDa. In another aspect, the number of repeating units is about 2 to about 500, e.g., about 2 to about 400, about 3 to about 300, about 3 to about 200, about 5 to about 100, about 7.5 to about 50, or about 10 to about 30. In another aspect, the repeating unit is any combination of G, X, L, F, S, T and J subunits, according to the nomenclature of Fry et al. (Physiologia Plantarum, 89: 1-3, 1993). In another aspect, the repeating unit is either fucosylated or non-fucosylated XXXG-type polymeric xyloglucan common to dicotyledons and nongraminaceous monocots. In another aspect, the polymeric xyloglucan is O-acetylated. In another aspect the polymeric xyloglucan is not O-acetylated. In another aspect, side chains of the polymeric xyloglucan may contain terminal fucosyl residues. In another aspect, side chains of the polymeric xyloglucan may contain terminal arabinosyl residues. In another aspect, side chains of the polymeric xyloglucan may contain terminal xylosyl residues.

For purposes of the present invention, references to the term xyloglucan herein refer to polymeric xyloglucan.

### Xyloglucan Oligomer

In the methods of the present invention, the xyloglucan oligomer can be any xyloglucan oligomer. The xyloglucan oligomer may be obtained by degradation or hydrolysis of polymeric xyloglucan from any source. The xyloglucan oligomer may be obtained by enzymatic degradation of polymeric xyloglucan, e.g., by quantitative or partial digestion with a xyloglucanase or endoglucanase (endo-β-1-4-glucanase). The xyloglucan oligomer may be synthesized from component carbohydrates, UDP- or GDP-carbohydrates, or halogenated carbohydrates by any of the manners commonly used by those skilled in the art.

In one aspect, the average molecular weight of the xyloglucan oligomer ranges from 0.5 kDa to about 500 kDa, e.g., about 1 kDa to about 20 kDa, about 1 kDa to about 10 kDa, or about 1 kDa to about 3 kDa. In another aspect, the number of repeating units is about 1 to about 500, e.g., about 1 to about 20, about 1 to about 10, or about 1 to about 3. In the methods of the present invention, the xyloglucan oligomer is optimally as short as possible (i.e., 1 repeating unit, or about 1 kDa in molecular weight) to maximize the solubility and solution molarity per gram of dissolved xyloglucan oligomer, while maintaining substrate specificity for xyloglucan endotransglycosylase activity. In another aspect, the xyloglucan oligomer comprises any combination of G (β-D glucopyranosyl-), X (α-D-xylopyranosyl-(1→6)-β-D-glucopyranosyl-), L (β-D-galactopyranosyl-(1→2)-α-D-xylopyranosyl-(1→6)-β-D-glucopyranosyl-), F (α-L-fuco-pyranosyl-(1→2)-β-D-galactopyranosyl-(1→2)-α-D-xylopyranosyl-(1→6)-β-D-glucopyranosyl-), S (α-L-arabinofurosyl-(1→2)-α-D-xylopyranosyl-(1→6)-β-D-glucopyranosyl-), T (α-L-arabino-furosyl-(1→3)-α-L-arabinofurosyl-(1→2)-α-D-xylopyranosyl-(1→6)-β-D-glucopyranosyl-), and J (α-L-galactopyranosyl-(1→2)-β-D-galactopyranosyl-(1→2)-α-D-xylopyranosyl-(1→6)-β-D-gluco-pyranosyl-) subunits according to the nomenclature of Fry et al. (Physiologia Plantarum 89: 1-3, 1993). In another aspect, the xyloglucan oligomer is the XXXG heptasaccharide common to dicotyledons and nongraminaceous monocots. In another aspect, the xyloglucan oligomer is O-acetylated. In another aspect, the xyloglucan oligomer is not O-acetylated. In another aspect, side chains of the xyloglucan oligomer may contain terminal fucosyl residues. In another aspect, side chains of the xyloglucan oligomer may contain terminal arabinosyl residues. In another aspect, side chains of the xyloglucan oligomer may contain terminal xylosyl residues.

### Functionalization of Xyloglucan Oligomer and Polymeric Xyloglucan

The xyloglucan oligomer can be functionalized by incorporating any chemical group known to those skilled in the art. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

In one aspect, the chemical group is an aldehyde group.

In another aspect, the chemical group is an amino group. The amino group can be an aliphatic amine or an aromatic amine (e.g., aniline). The amine can be a primary, secondary or tertiary amine.

In another aspect, the chemical group is an aromatic group. The aromatic group can be an arene group, an aryl halide group, a phenolic group, a phenylamine group, a diazonium group, or a heterocyclic group.

In another aspect, the chemical group is a carboxyl group. The carboxyl group can be an acyl halide, an amide, a carboxylic acid, an ester, or a thioester.

In another aspect, the chemical group is a halogen group. The halogen group can be fluorine, chlorine, bromine, or iodine.

In another aspect, the chemical group is a hydroxyl group.

In another aspect, the chemical group is a ketone group.

In another aspect, the chemical group is a nitrile group.

In another aspect, the chemical group is a nitro group.

In another aspect, the chemical group is a sulfhydryl group.

In another aspect, the chemical group is a sulfonate group.

The chemical reactive group can itself be the chemical group that imparts a desired physical or chemical property to a filler material.

By incorporation of chemical reactive groups in such a manner, one skilled in the art can further derivatize the incorporated reactive groups with compounds (e.g., macromolecules) that will impart a desired physical or chemical property to a filler material. The derivatization can be performed directly on the functionalized xyloglucan oligomer or after the functionalized xyloglucan oligomer is incorporated into polymeric xyloglucan.

Alternatively, the xyloglucan oligomer can be functionalized by incorporating directly a compound that imparts a desired physical or chemical property to a filler material by using a reactive group contained in the compound or a reactive group incorporated into the compound, such as any of the groups described above.

On the other hand, the polymeric xyloglucan can be directly functionalized by incorporating a reactive group or a chemical compound as described above. By incorporation of chemical reactive groups directly into polymeric xyloglucan, one of skill in the art can further derivatize the incorporated reactive groups with compounds that will impart a desired physical or chemical property to a material. By incorporation of a compound directly into the polymeric xyloglucan, a desired physical or chemical property can also be directly imparted to a material.

In one aspect, the functionalization is performed by reacting the reducing end hydroxyl of the xyloglucan oligomer or the polymeric xyloglucan. In another aspect, a non-reducing hydroxyl group, other than the non-reducing hydroxyl at position 4 of the terminal glucose, can be reacted. In another aspect, the reducing end hydroxyl and a non-reducing hydroxyl, other than the non-reducing hydroxyl at position 4 of the terminal glucose, can be reacted.

The chemical functional group can be added by enzymatic modification of the xyloglucan oligomer or polymeric xyloglucan, or by a non-enzymatic chemical reaction. In one aspect, enzymatic modification is used to add the chemical functional group. In one embodiment of enzymatic modification, the enzymatic functionalization is oxidation to a ketone or carboxylate, e.g., by galactose oxidase. In another embodiment of enzymatic modification, the enzymatic functionalization is oxidation to a ketone or carboxylate by AA9 Family oxidases (formerly glycohydrolase Family 61 enzymes).

In another aspect, the chemical functional group is added by a non-enzymatic chemical reaction. In one embodiment of the non-enzymatic chemical reaction, the reaction is reductive amination of the reducing end of the carbohydrate as described by Roy et al., 1984, Can. J. Chem. 62: 270-275, or Dalpathado et al., 2005, Anal. Bioanal. Chem. 381: 1130-1137. In another embodiment of non-enzymatic chemical reaction, the reaction is oxidation of the reducing end hydroxyl to a ketone, e.g., by copper (II). In another embodiment of non-enzymatic chemical reaction, the reaction is oxidation of non-reducing end hydroxyl groups (e.g., of the non-glycosidic bonded position 6 hydroxyls of glucose or galactose) by (2,2,6,6-tetramethyl-piperidin-1-yl)oxyl (TEMPO), or the oxoammonium salt thereof, to generate an aldehyde or carboxylic acid as described in Bragd et al., 2002, Carbohydrate Polymers 49: 397-406, or Breton et al., 2007, Eur. J. Org. Chem. 10: 1567-1570.

Xyloglucan oligomers or polymeric xyloglucan can be functionalized by a chemical reaction with compounds containing more than one (i.e. bifunctional or multifunctional) chemical functional group comprising at least one chemical functional group that is directly reactive with xyloglucan oligomer or polymeric xyloglucan. In one aspect, the bifunctional chemical group is a hydrocarbon containing a primary amine and a second functional group. The second functional group can be any of the other groups described above.

Xyloglucan oligomers or polymeric xyloglucan can be functionalized with a compound of interest by step-wise or concerted reaction wherein the xyloglucan oligomer or polymeric xyloglucan is functionalized as described above, and the compound is reactive to the functionalization introduced therein. In one aspect of coupling via a functionalized xyloglucan oligomer, an amino group is first incorporated into the xyloglucan oligomer by reductive amination and a reactive carbonyl is secondarily coupled to the introduced amino group. In another aspect of coupling via an amino-modified xyloglucan oligomer, the second coupling step incorporates a chemical group, compound or macromolecule via coupling an N-hydroxysuccinimidyl (NHS) ester or imidoester to the introduced amino group. In a preferred embodiment, the NHS ester secondarily coupled to the introduced amino group is a component of a mono or bi-functional crosslink reagent. In another aspect of coupling to a functionalized xyloglucan or xyloglucan oligomer, the first reaction step comprises functionalization with a sulfhydryl group, either via reductive amination with an alkylthioamine (NH₂—(CH₂)ₙ—SH) at elevated temperatures in the presence of a reducing agent (Magid et al., 1996, J. Org. Chem. 61: 3849-3862), or via radical coupling (Wang et al., 2009, Arkivoc xiv: 171-180), followed by reaction of a maleimide group to the sulfhydryl.

Non-limiting examples of compounds of interest that can be used to functionalize polymeric xyloglucan or xyloglucan oligomers, either by direct reaction or via reaction with a xyloglucan-reactive compound, include peptides, polypeptides, proteins, hydrophobic groups, hydrophilic groups, flame retardants, dyes, color modifiers, specific affinity tags, non-specific affinity tags, metals, metal oxides, metal sulfides, minerals, fungicides, herbicides, microbicides or microbiostatics, and non-covalent linker molecules.

In one aspect, the compound is a peptide. The peptide can be an antimicrobial peptide, a “self-peptide” designed to reduce allergenicity and immunogenicity, a cyclic peptide, glutathione, or a signaling peptide (such as a tachykinin peptide, vasoactive intestinal peptide, pancreatic polypeptide related peptide, calcitonin peptide, lipopeptide, cyclic lipopeptide, or other peptide).

In another aspect, the compound is a polypeptide. The polypeptide can be a non-catalytically active protein (i.e., structural or binding protein) or a catalytically active protein (i.e., enzyme). The polypeptide can be an enzyme, an antibody, or an abzyme.

In another aspect, the compound is a hydrophobic group. The hydrophobic group can be polyurethane, polytetrafluoroethylene, or polyvinylidene fluoride.

In another aspect, the compound is a hydrophilic group. The hydrophilic group can be methacylate, methacrylamide, or polyacrylate.

In another aspect, the compound is a flame retardant. The flame-retardant can be aluminum hydroxide or magnesium hydroxide. The flame-retardant can also be an organohalogen group or an organophosphorous group.

In another aspect, the compound is a dye or pigment group.

In another aspect, the compound is a specific affinity tag. The specific affinity tag can be biotin, avidin, a chelating group, a crown ether, a heme group, a non-reactive substrate analog, an antibody, target antigen, or a lectin.

In another aspect, the compound is a non-specific affinity tag. The non-specific affinity tag can be a polycation group, a polyanion group, a magnetic particle (e.g., magnetite), a hydrophobic group, an aliphatic group, a metal, a metal oxide, a metal sulfide, or a molecular sieve.

In another aspect, the compound is a fungicide. The fungicide can be a dicarboximide group (such as vinclozolin), a phenylpyrrole group (such as fludioxonil), a chlorophenyl group (such as quintozene), a chloronitrobenzene (such as dicloran), a triadiazole group (such as etridiazole), a dithiocarbamate group (such as mancozeb or dimethyldithiocarbamate), or an inorganic molecule (such as copper or sulfur). In another aspect, the fungicide is a bacteria or bacterial spore such as Bacillus.

In another aspect, the compound is a herbicide. The herbicide can be glyphosate, a synthetic plant hormone (such as a 2,4-dichloropenoxyacetic acid group, a 2,4,5-trichlorophenoxyacetic acid group, a 2-methyl-4-chlorophenoxyacetic acid group, a 2-(2-methyl-4-chlorophenoxy)propionic acid group, a 2-(2,4-dichlorophenoxy)propionic acid group, or a (2,4-dichlorophenoxy)butyric acid group), or a triazine group (such as atrazine (2-chloro-4-(ethylamino)-6-isopropylamino)-s-triazine).

In another aspect, the compound is a bactericidal or bacteriostatic compound. The bactericidal or bacteriostatic compound can be a copper or copper alloy (such as brass, bronze, cupronickel, or copper-nickel-zinc alloy), a sulfonamide group (such as sulfamethoxazole, sulfisomidine, sulfacetamide or sulfadiazine), a silver or organo-silver group, TiO₂, ZnO₂, an antimicrobial peptide, or chitosan.

In another aspect, the compound is a non-covalent linker molecule. In another aspect, the compound is a color modifier. The color modifier can be a dye, fluorescent brightener, color modifier, or mordant (e.g., alum, chrome alum).

In another aspect, the compound is a metal.

In another aspect, the compound is a semi-conductor. The semi-conductor can be an organic semi-conductor, a binary or ternary compound, or a semi-conducting element.

In another aspect, the compound is a UV-resistant compound. The UV resistant compound can be zinc or ZnO₂, kaolin, aluminum, aluminum oxides, or other UV-resistant compounds.

In another aspect, the compound is an anti-oxidant compound. The anti-oxidant compound can be ascorbate, retinol, tocopherol, manganese, iodide, a terpenoid, a flavonoid or other anti-oxidant phenolic or polyphenolic or other anti-oxidant compounds.

### Preparation of Modified Filler Materials

In the processes of the present invention, a modified filler material can be prepared from any filler material known in the art. The filler material can be modified by treating a suspension of the filler material with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; or (i) a composition of (a), (b), (c), (d), (e), (f), (g), or (h) without a xyloglucan endotransglycosylase, under conditions leading to a modified filler material, wherein the modified filler material possesses an improved property compared to the unmodified filler material.

The processes of the present inventiom are exemplified below by functionalization of titanium dioxide with a fluorescent dye, thereby imparting desired optical properties to the filler material. However, the filler material can also be kaolin, silicon dioxide or any other filler material known in the art. A slurry of titanium dioxide can be incubated in a pH controlled solution, e.g., buffered solution (e.g., sodium citrate) from pH 3 to pH 9, e.g., pH 4 to pH 8 or pH 5 to pH 7, at concentrations from about 1 g/L to about 10 kg/L, e.g., about 10 g/L to about 1 kg/L or about 40 g/L to about 100 g/L containing xyloglucan endotransglycosylase and polymeric xyloglucan with or without functionalized xyloglucan oligomer. The xyloglucan endotransglycosylase can be present at about 0.1 nM to about 1 mM, e.g., about 10 nM to about 100 μM or about 0.5 μM to about 5 μM. In one aspect, the xyloglucan endotransglycosylase is present at a concentration of 320 pg to about 32 mg of enzyme per g of the filler material, e.g., about 160 μg to about 4 mg of enzyme per g of the filler material. When present, the molar ratio of functionalized xyloglucan oligomer to polymeric xyloglucan is about 50:1 molar ratio to about 0.5:1, e.g., about 10:1 to about 1:1 or about 5:1 to about 1:1. The polymeric xyloglucan can be present at about 1 mg per g of the filler material to about 1 g per g of the filler material, e.g., about 10 mg to about 100 mg per g of the filler material or about 20 mg to about 50 mg per g of the filler material. The incubation can last for a sufficient period of time as to effect the desired extent of functionalization, e.g., about instantaneously to about 72 hours, about 15 minutes to about 48 hours, about 30 minutes to about 24 hours, or about 1 hour to about 3 hours at room temperature. The temperature and incubation time can be optimized by one skilled in the art. The filler material can then be separated from xyloglucan endotransglycosylase and unbound polymeric xyloglucan or functionalized xyloglucan oligomer by washing, for example, in water. In another aspect of the present invention, the filler material is then dried.

In one aspect of the present invention, the polymeric xyloglucan is functionalized prior to modification of the filler materials. The polymeric xyloglucan can be incubated in pH controlled solution with xyloglucan endotransglycosylase and functionalized xyloglucan oligomers, yielding functionalized xyloglucan. Functionalized xyloglucan can then be separated from functionalized xyloglucan oligomers by any method known to those skilled in the art, but as exemplified by ethanol precipitation. For example, the reaction mixture can be incubated in 80% (v/v) ethanol for about 1 minute to about 24 hours, e.g., 30 minutes to 20 hours or 1 to 15 hours, centrifuged for an appropriate length of time at an appropriate velocity to pellet the precipitated, functionalized xyloglucan (e.g., 30 minutes at approximately 2000×g), and the supernatants decanted off. The functionalized xyloglucan is then optionally dried. In this aspect of the present invention, the functionalized xyloglucan is then incubated with xyloglucan endotransglycosylase and the filler material in an aqueous suspension. Contingent upon the product, e.g., grade of paper or board produced, mineral fillers can comprise 1-30% of the final weight of the product. Fillers are generally added without any modification of process conditions within the stock preparation operations or the paper or board machine. However, in separate embodiments, the modified filler can be added in the presence or absence of conventional retention programs.

### Sources of Xyloglucan Endotransglycosylases

Any xyloglucan endotransglycosylase may be used that possesses suitable enzyme activity at a pH and temperature appropriate for the methods of the present invention. It is preferable that the xyloglucan endotransglycosylase is active over a broad pH and temperature range. In an embodiment, the xyloglucan endotransglycosylase has a pH optimum in the range of about 3 to about 10. In another embodiment, the xyloglucan endotransglycosylase has a pH optimum in the range of about 4.5 to about 8.5. In another embodiment, the xyloglucan endotransglycosylase has a cold denaturation temperature less than or equal to about 5° C. or a melting temperature of about 100° C. or higher. In another embodiment, the xyloglucan endotransglycosylase has a cold denaturation temperature of less than or equal to 20° C. or a melting temperature greater than or equal to about 75° C.

The source of the xyloglucan endotransglycosylase used is not critical in the present invention. Accordingly, the xyloglucan endotransglycosylase may be obtained from any source such as a plant, microorganism, or animal.

In one embodiment, the xyloglucan endotransglycosylase is obtained from a plant source. Xyloglucan endotransglycosylase can be obtained from cotyledons of the family Fabaceae (synonyms: Leguminosae and Papilionaceae), preferably genus Phaseolus, in particular, Phaseolus aureus. Preferred monocotyledons are non-graminaceous monocotyledons and liliaceous monocotyledons. Xyloglucan endotransglycosylase can also be extracted from moss and liverwort, as described in Fry et al., 1992, Biochem. J. 282: 821-828. For example, the xyloglucan endotransglycosylase may be obtained from cotyledons, i.e., a dicotyledon or a monocotyledon, in particular a dicotyledon selected from the group consisting of azuki beans, cauliflowers, cotton, poplar or hybrid aspen, potatoes, rapes, soy beans, sunflowers, thalecress, tobacco, and tomatoes, or a monocotyledon selected from the group consisting of wheat, rice, corn, and sugar cane. See, for example, WO 2003/033813 and WO 97/23683.

In another embodiment, the xyloglucan endotransglycosylase is obtained from Arabidopsis thaliana (GENESEQP:AOE11231, GENESEQP:AOE93420, GENESEQP: BAL03414, GENESEQP:BAL03622, or GENESEQP:AWK95154); Carica papaya (GENESEQP:AZR75725); Cucumis sativus (GENESEQP:AZV66490); Daucus carota (GENESEQP:AZV66139); Festuca pratensis (GENESEQP:AZR80321); Glycine max (GENESEQP:AWK95154 or GENESEQP:AYF92062); Hordeum vulgare (GENESEQP:AZR85056, GENESEQP:AQY12558, GENESEQP:AQY12559, or GENESEQP:AWK95180); Lycopersicon esculentum (GENESEQP:ATZ45232); Medicago truncatula (GEN ES EQP:ATZ48025); Oryza sativa (GENESEQP:ATZ42485, GENESEQP:ATZ57524, or GENESEQP:AZR76430); Populus tremula (GENESEQP:AWK95036); Sagittaria pygmaea (GENESEQP:AZV66468); Sorghum bicolor (GENESEQP:BA079623 or GENESEQP:BA079007); Vigna angularis (GENESEQP:ATZ61320); or Zea mays (GENESEQP:AWK94916).

In another embodiment, the xyloglucan endotransglycosylase is a xyloglucan endotransglucosylase/hydrolase (XTH) with both hydrolytic and transglycosylating activities. In a preferred embodiment, the ratio of transglycosylation to hydrolytic rates is at least 10⁻² to 10⁷, e.g., 10⁻¹ to 10⁶ or 10 to 1000.

### Production of Xyloglucan Endotransglycosylases

Xyloglucan endotransglycosylase may be extracted from plants. Suitable methods for extracting xyloglucan endotransglycosylase from plants are described Fry et al., 1992, Biochem. J. 282: 821-828; Sulova et al., 1998, Biochem. J. 330: 1475-1480; Sulova et al., 1995, Anal. Biochem. 229: 80-85; WO 95/13384; WO 97/23683; or EP 562 836.

Xyloglucan endotransglycosylase may also be produced by cultivation of a transformed host organism containing the appropriate genetic information from a plant, microorganism, or animal. Transformants can be prepared and cultivated by methods known in the art.

Techniques used to isolate or clone a gene are known in the art and include isolation from genomic DNA or cDNA, or a combination thereof. The cloning of the gene from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

A nucleic acid construct can be constructed to comprise a gene encoding a xyloglucan endotransglycosylase operably linked to one or more control sequences that direct the expression of the coding sequence in a suitable host cell under conditions compatible with the control sequences. The gene may be manipulated in a variety of ways to provide for expression of the xyloglucan endotransglycosylase. Manipulation of the gene prior to its insertion into a vector may be desirable or necessary depending on the expression vector. Techniques for modifying polynucleotides utilizing recombinant DNA methods are well known in the art.

The control sequence may be a promoter, a polynucleotide that is recognized by a host cell for expression of a polynucleotide encoding a xyloglucan endotransglycosylase. The promoter contains transcriptional control sequences that mediate the expression of the xyloglucan endotransglycosylase. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryllIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dana (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the xyloglucan endotransglycosylase. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryllIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the xyloglucan endotransglycosylase. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a xyloglucan endotransglycosylase and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a xyloglucan endotransglycosylase. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a xyloglucan endotransglycosylase and the signal peptide sequence is positioned next to the N-terminus of the propeptide sequence.

The various nucleotide and control sequences may be joined together to produce a recombinant expression vector that may include one or more convenient restriction sites to allow for insertion or substitution of the polynucleotide encoding the xyloglucan endotransglycosylase at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the xyloglucan endotransglycosylase or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide may be inserted into a host cell to increase production of a xyloglucan endotransglycosylase. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

The host cell may be any cell useful in the recombinant production of a xyloglucan endotransglycosylase, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The host cells are cultivated in a nutrient medium suitable for production of the xyloglucan endotransglycosylase using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the xyloglucan endotransglycosylase to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the xyloglucan endotransglycosylase is secreted into the nutrient medium, the polypeptide can be recovered directly from the medium. If the xyloglucan endotransglycosylase is not secreted, it can be recovered from cell lysates.

The xyloglucan endotransglycosylase may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The xyloglucan endotransglycosylase may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a whole fermentation broth comprising the polypeptide is recovered. In a preferred aspect, xyloglucan endotransglycosylase yield may be improved by subsequently washing cellular debris in buffer or in buffered detergent solution to extract biomass-associated polypeptide.

The xyloglucan endotransglycosylase may be purified by a variety of procedures known in the art including, but not limited to, chromatography (e.g., ion exchange, affinity, hydrophobic interaction, mixed mode, reverse phase, chromatofocusing, and size exclusion), electrophoretic procedures (e.g., preparative isoelectric focusing), differential solubility (e.g., ammonium sulfate precipitation), PAGE, membrane-filtration or extraction (see, e.g., Protein Purification, Janson and Ryden, editors, VCH Publishers, New York, 1989) to obtain substantially pure polypeptide. In a preferred aspect, xyloglucan endotransglycosylase may be purified by formation of a covalent acyl-enzyme intermediate with xyloglucan, followed by precipitation with microcrystalline cellulose or adsorption to cellulose membranes. Release of the polypeptide is then effected by addition of xyloglucan oligomers to resolve the covalent intermediate (Sulova and Farkas, 1999, Protein Expression and Purification 16(2): 231-235, and Steele and Fry, 1999, Biochemical Journal 340: 207-211).

The present invention is further described by the following examples that should not be construed as limiting the scope of the invention.

### EXAMPLES

### Media and Solutions

COVE agar plates were composed of 342.3 g of sucrose, 252.54 g of CsCl, 59.1 g of acetamide, 520 mg of KCl, 520 mg of MgSO₄.7H₂O, 1.52 g of KH₂PO₄, 0.04 mg of Na₂B₄O₇.10H₂O, 0.4 mg of CuSO₄.5H₂O, 1.2 mg of FeSO₄.7H₂O, 0.7 mg of MnSO₄.2H₂O, is 0.8 mg of Na₂MoO₄.2H₂O, 10 mg of ZnSO₄.7H₂O, 25 g of Noble agar, and deionized water to 1 liter.

LB medium was composed of 10 g of tryptone, 5 g of yeast extract, 5 g of NaCl, and deionized water to 1 liter.

LB plates were composed of 10 g of tryptone, 5 g of yeast extract, 5 g of NaCl, 15 g of bacteriological agar, and deionized water to 1 liter.

Minimal medium agar plates were composed of 342.3 g of sucrose, 10 g of glucose, 4 g of MgSO_{4.}7H₂0, 6 g of NaNO₃, 0.52 g of KCl, 1.52 g of KH₂PO₄, 0.04 mg of Na₂B₄O₇.10H₂O, 0.4 mg of CuSO₄.5H₂O, 1.2 mg of FeSO₄.7H₂O, 0.7 mg of MnSO₄.2H₂O, 0.8 mg of Na₂MoO₄.2H₂O, 10 mg of ZnSO₄.7H₂O, 500 mg of citric acid, 4 mg of d-biotin, 20 g of Noble agar, and deionized water to 1 liter.

Synthetic Defined medium lacking uridine was composed of 18 mg of adenine hemisulfate, 76 mg of alanine, 76 mg of arginine hydrochloride, 76 mg of asparagine monohydrate, 76 mg of aspartic acid, 76 mg of cysteine hydrochloride monohydrate, 76 mg of glutamic acid monosodium salt, 76 mg of glutamine, 76 mg of glycine, 76 mg of histidine, myo-76 mg of inositol, 76 mg of isoleucine, 380 mg of leucine, 76 mg of lysine monohydrochloride, 76 mg of methionine, 8 mg of p-aminobenzoic acid potassium salt, 76 mg of phenylalanine, 76 mg of proline, 76 mg of serine, 76 mg of threonine, 76 mg of tryptophan, 76 mg of tyrosine disodium salt, 76 mg of valine, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

TBE buffer was composed of 10.8 g of Tris Base, 5.5 g of boric acid, 4 ml of 0.5 M EDTA pH 8.0, and deionized water to 1 liter.

2XYT plus ampicillin plates were composed of 16 g of tryptone, 10 g of yeast extract, 5 g of sodium chloride, 15 g of Bacto agar, and deionized water to 1 liter. One ml of a 100 mg/ml solution of ampicillin was added after the autoclaved medium was tempered to 55° C.

YP+2% glucose medium was composed of 10 g of yeast extract, 20 g of peptone, 20 g of glucose, and deionized water to 1 liter.

YP+2% maltodextrin medium was composed of 10 g of yeast extract, 20 g of peptone, 20 g of maltodextrin, and deionized water to 1 liter.

### Example 1

### Preparation of Vigna angularis Xyloglucan Endotransglycosylase 16

Vigna angularis xyloglucan endotransglycosylase 16 (VaXET16; SEQ ID NO: 1 [native DNA sequence], SEQ ID NO: 2 [synthetic DNA sequence], and SEQ ID NO: 3 [deduced amino acid sequence]; also referred to as XTH1) was recombinantly produced in Aspergillus oryzae MT3568 according to the protocol described below. Aspergillus oryzae MT3568 is an amdS (acetamidase) disrupted gene derivative of Aspergillus oryzae JaL355 (WO 2002/40694), in which pyrG auxotrophy was restored by disrupting the A. oryzae acetamidase (amdS) gene with the pyrG gene.

The vector pDLHD0012 was constructed to express the VaXET16 gene in multi-copy in Aspergillus oryzae. Plasmid pDLHD0012 was generated by combining two DNA fragments using megaprimer cloning: fragment 1 containing the VaXET16 ORF and flanking sequences with homology to vector pBM120 (US20090253171), and fragment 2 consisting of an inverse PCR amplicon of vector pBM120.

Fragment 1 was amplified using primer 613788 (sense) and primer 613983 (antisense) shown below. These primers were designed to contain flanking regions of sequence homology to vector pBM120 (lower case) for ligation-free cloning between the PCR fragments.

Primer 613788 (sense):

(SEQ ID NO: 7)

ttcctcaatcctctatatacacaactggccATGGGCTCGTCCCTCTGGAC
Primer 613983 (antisense):

(SEQ ID NO: 8)

tgtcagtcacctctagttaattaGATGTCCCTATCGCGTGTACACTCG

Fragment 1 was amplified by PCR in a reaction composed of 10 ng of a GENEART® vector pMA containing the VaXET16 synthetic gene (SEQ ID NO: 3 [synthetic DNA sequence]) cloned between the Sac I and Kpn I sites, 0.5 μl of PHUSION® DNA Polymerase (New England Biolabs, Inc., Ipswich, Mass., USA), 20 pmol of primer 613788, 20 pmol of primer 613983, 1 μl of 10 mM dNTPs, 10 μl of 5× PHUSION® HF buffer (New England Biolabs, Inc., Ipswich, Mass., USA), and 35.5 μl of water. The reaction was incubated in an EPPENDORF® MASTERCYCLER® (Eppendorf AG, Hamburg, Germany) programmed for 1 cycle at 98° C. for 30 seconds; and 30 cycles each at 98° C. for 10 seconds, 60° C. for 10 seconds, and 72° C. for 30 seconds. The resulting 0.9 kb PCR product (fragment 1) was treated with 1 μl of Dpn I (Promega, Fitchburg, Wis., USA) to remove plasmid template DNA. The Dpn I was added directly to the PCR reaction tube, mixed well, and incubated at 37° C. for 60 minutes, and then was column-purified using a MINELUTE® PCR Purification Kit (QIAGEN Inc., Valencia, Calif., USA) according to the manufacturer's instructions.

Fragment 2 was amplified using primers 613786 (sense) and 613787 (antisense) shown below.

613786 (sense):

(SEQ ID NO: 9)

taattaactagaggtgactgacacctggc
613787 (antisense):

(SEQ ID NO: 10)

catggccagttgtgtatatagaggattgagg

Fragment 2 was amplified by PCR in a reaction composed of 10 ng of plasmid pBM120, 0.5 μl of PHUSION® DNA Polymerase, 20 pmol of primer 613786, 20 pmol of primer 613787, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 6.9 kb PCR product (fragment 2) was treated with 1 μl of Dpn I to remove plasmid template DNA. The Dpn I was added directly to the PCR reaction tube, mixed well, and incubated at 37° C. for 60 minutes, and then column-purified using a MINELUTE® PCR Purification Kit according to the manufacturer's instructions.

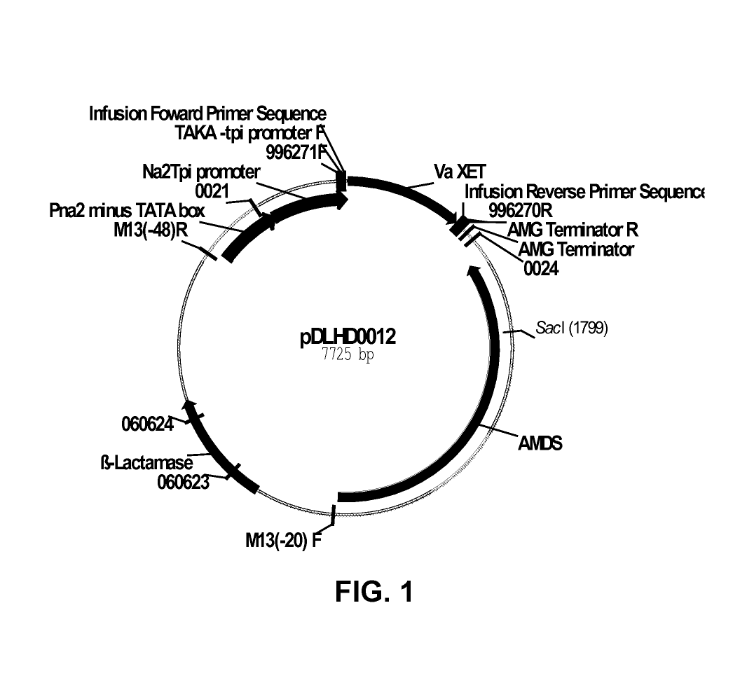

The following procedure was used to combine the two PCR fragments using megaprimer cloning. Fragments 1 and 2 were combined by PCR in a reaction composed of 5 μl of each purified PCR product, 0.5 μl of PHUSION® DNA Polymerase, 1 μl of 10 mM dNTPs, 10 μl of 5× PHUSION® HF buffer, and 28.5 μl of water. The reaction was incubated in an EPPENDORF® MASTERCYCLER® programmed for 1 cycle at 98° C. for 30 seconds; and 40 cycles each at 98° C. for 10 seconds, 60° C. for 10 seconds, and 72° C. for 4 minutes. Two μl of the resulting PCR product DNA was then transformed into E. coli ONE SHOT® TOP10 electrocompetent cells (Life Technologies, Grand Island, N.Y., USA) according the manufacturer's instructions. Fifty μl transformed cells were spread onto LB plates supplemented with 100 μg of ampicillin per ml and incubated at 37° C. overnight. Individual transformants were picked into 3 ml of LB medium supplemented with 100 μg of ampicillin per ml and grown overnight at 37° C. with shaking at 250 rpm. The plasmid DNA was purified from the colonies using a QIAPREP® Spin Miniprep Kit (QIAGEN Inc., Valencia, Calif., USA). DNA sequencing using a 3130XL Genetic Analyzer (Applied Biosystems, Foster City, Calif., USA) was used to confirm the presence of each of both fragments in the final plasmid pDLHD0012 (FIG. 1).

Aspergillus oryzae strain MT3568 was transformed with plasmid pDLHD0012 comprising the VaXET16 gene according to the following protocol. Approximately 2-5×10⁷ spores of A. oryzae strain MT3568 were inoculated into 100 ml of YP+2% glucose medium in a 500 ml shake flask and incubated at 28° C. and 110 rpm overnight. Ten ml of the overnight culture were filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCl-20 mM CaCl₂ and transferred to a sterile 125 ml shake flask containing 20 mg of GLUCANEX® 200 G (Novozymes Switzerland AG, Neumatt, Switzerland) per ml and 0.2 mg of chitinase (Sigma-Aldrich, St. Louis, Mo., USA) per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® (Calbiochem, San Diego, Calif., USA) into a sterile 50 ml plastic centrifuge tube to remove mycelial debris. The debris in the MIRACLOTH® was washed thoroughly with 0.7 M KCl-20 mM CaCl₂, and centrifuged at 2500 rpm (537×g) for 10 minutes at 20-23° C. The supernatant was removed and the protoplast pellet was resuspended in 20 ml of 1 M sorbitol-10 mM Tris-HCl (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0012 were added to the bottom of a sterile 2 ml plastic centrifuge tube. Then 100 μl of protoplasts were added to the tube followed by 300 ×l of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm COVE agar plates. Transformation plates were incubated at 34° C. until transformants appeared.

Twenty-one transformants were picked to fresh COVE agar plates and cultivated at 34° C. for four days until the transformants sporulated. Fresh spores were transferred to 48-well deep-well plates containing 2 ml of YP+2% maltodextrin, covered with a breathable seal, and grown for 4 days at 34° C. with no shaking. After 4 days growth samples of the culture media were assayed for xyloglucan endotransglycosylase activity using an iodine stain assay and for xyloglucan endotransglycosylase expression by SDS-PAGE.

The iodine stain assay for xyloglucan endotransglycosylase activity was performed according to the following protocol. In a 96-well plate, 5 μl of culture broth was added to a mixture of 5 μl of xyloglucan (Megazyme, Bray, United Kingdom) (5 mg/ml in water), 20 μl of xyloglucan oligomers (Megazyme, Bray, United Kingdom) (5 mg/ml in water), and 10 μl of 400 mM sodium citrate pH 5.5. The reaction mix was incubated at 37° C. for thirty minutes, quenched with 200 μl of a solution containing 14% (w/v) Na₂SO₄, 0.2% KI, 100 mM HCI, and 1% iodine (1₂), incubated in the dark for 30 minutes, and then the absorbance was measured in a plate reader at 620 nm. The assay demonstrated the presence of xyloglucan endotransglycosylase activity from several transformants.

SDS-PAGE was performed using a 8-16% CRITERION® Stain Free SDS-PAGE gel (Bio-Rad Laboratories, Inc., Hercules, Calif., USA), and imaging the gel with a Stain Free Imager (Bio-Rad Laboratories, Inc., Hercules, Calif., USA) using the following settings: 5-minute activation, automatic imaging exposure (intense bands), highlight saturated pixels=ON, color=Coomassie, and band detection, molecular weight analysis and reporting disabled. SDS-PAGE revealed a band of approximately 32 kDa corresponding to VaXET16 in several transformants.

### Example 2

### Construction of Plasmid pMMar27 as a Yeast Expression Plasmid Vector

Plasmid pMMar27 was constructed for expression of the T. terrestris Cel6A cellobiohydrolase II in yeast. The plasmid was generated from a lineage of yeast expression vectors: plasmid pMMar27 was constructed from plasmid pBM175b; plasmid pBM175b was constructed from plasmid pBM143b (WO 2008/008950) and plasmid pJLin201; and plasmid pJLin201 was constructed from pBM143b.

Plasmid pJLin201 is identical to pBM143b except an Xba I site immediately downstream of a Thermomyces lanuginosus lipase variant gene in pBM143b was mutated to a unique Nhe I site. A QUIKCHANGE® II XL Site-Directed Mutagenesis Kit (Stratagene, La Jolla, Calif., USA) was used to change the Xba I sequence (TCTAGA) to a Nhe I sequence (gCTAGc) in pBM143b. Primers employed to mutate the site are shown below.

Primer 999551 (sense):

(SEQ ID NO: 11)

5′-ACATGTCTTTGATAAgCTAGcGGGCCGCATCATGTA-3′
Primer 999552 (antisense):

(SEQ ID NO: 12)

5′-TACATGATGCGGCCCgCTAGcTTATCAAAGACATGT-3′
Lower case represents mutated nucleotides.

The amplification reaction was composed of 125 ng of each primer above, 20 ng of pBM143b, 1× QUIKCHANGE® Reaction Buffer (Stratagene, La Jolla, Calif., USA), 3 μl of QUIKSOLUTION® (Stratagene, La Jolla, Calif., USA), 1 μl of dNTP mix, and 1 μl of a 2.5 units/ml Pfu Ultra HF DNA polymerase, in a final volume of 50 μl. The reaction was performed using an EPPENDORF® MASTERCYCLER® thermocycler programmed for 1 cycle at 95° C. for 1 minute; 18 cycles each at 95° C. for 50 seconds, 60° C. for 50 seconds, and 68° C. for 6 minutes and 6 seconds; and 1 cycle at 68° C. for 7 minutes. After the PCR reaction, the tube was placed on ice for 2 minutes. One microliter of Dpn I was directly added to the amplification reaction and incubated at 37° C. for 1 hour. A 2 μl volume of the Dpn I digested reaction was used to transform E. coli XL10-GOLD® Ultracompetent Cells (Stratagene, La Jolla, Calif., USA) according to the manufacturer's instructions. E. coli transformants were selected on 2XYT plus ampicillin plates. Plasmid DNA was isolated from several of the transformants using a BIOROBOT® 9600. One plasmid with the desired Nhe I change was confirmed by restriction digestion and sequencing analysis and designated plasmid pJLin201. To eliminate possible PCR errors introduced by site-directed-mutagenesis, plasmid pBM175b was constructed by cloning the Nhe I site containing fragment back into plasmid pBM143b. Briefly, plasmid pJLin201 was digested with Nde I and Mu I and the resulting fragment was cloned into pBM143b previously digested with the same enzymes using a Rapid Ligation Kit (Roche Diagnostics Corporation, Indianapolis, Ind., USA). Briefly, 7 μl of the Nde I/M/u I digested pJLin201 fragment and 1 μl of the digested pBM143b were mixed with 2 μl of 5× DNA dilution buffer (Roche Diagnostics Corporation, Indianapolis, Ind., USA), 10 μl of 2× T4 DNA Ligation buffer (Roche Diagnostics Corporation, Indianapolis, Ind., USA), and 1 μl of T4 DNA ligase (Roche Diagnostics Corporation, Indianapolis, Ind., USA) and incubated for 15 minutes at room temperature. Two microliters of the ligation were transformed into XL1-Blue Subcloning-Grade Competent Cells (Stratagene, La Jolla, Calif., USA) cells and spread onto 2XYT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pBM175b.

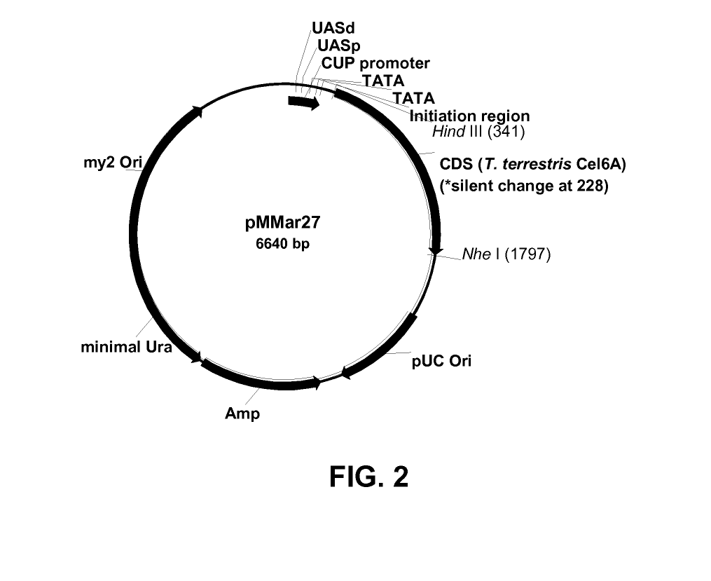

Plasmid pMMar27 was constructed from pBM175b and an amplified gene of T. terrestris Cel6A cellobiohydrolase II with overhangs designed for insertion into digested pBM175b. Plasmid pBM175b containing the Thermomyces lanuginosus lipase variant gene under control of the CUP I promoter contains unique Hind III and Nhe I sites to remove the lipase gene. Plasmid pBM175 was digested with these restriction enzymes to remove the lipase gene. After digestion, the empty vector was isolated by 1.0% agarose gel electrophoresis using TBE buffer where an approximately 5,215 bp fragment was excised from the gel and extracted using a QIAQUICK® Gel Extraction Kit. The ligation reaction (20 μl) was composed of 1× IN-FUSION® Buffer (BD Biosciences, Palo Alto, Calif., USA), 1× BSA (BD Biosciences, Palo Alto, Calif., USA), 1 p1 of IN-FUSION® enzyme (diluted 1:10) (BD Biosciences, Palo Alto, Calif., USA), 99 ng of pBM175b digested with Hind III and Nhe I, and 36 ng of the purified T. terrestris Cel6A cellobiohydrolase II PCR product. The reaction was incubated at room temperature for 30 minutes. A 2 μl volume of the IN-FUSION® reaction was transformed into E. coli XL10-GOLD® Ultracompetent Cells. Transformants were selected on LB plates supplemented with 100 μg of ampicillin per ml. A colony was picked that contained the T. terrestris Cel6A inserted into the pBM175b vector in place of the lipase gene, resulting in pMMar27 (FIG. 2). The plasmid chosen contained a PCR error at position 228 from the start codon, TCT instead of TCC, but resulted in a silent change in the T. terrestris Cel6A cellobiohydrolase II.

### Example 3

### Construction of pEvFz1 Expression Vector

Expression vector pEvFz1 was constructed by modifying pBM120a (U.S. Pat. No. 8,263,824) to comprise the NA2/NA2-tpi promoter, A. niger amyloglucosidase terminator sequence (AMG terminator), and Aspergillus nidulans orotidine-5′-phosphate decarboxylase gene (pyrG) as a selectable marker.

Plasmid pEvFz1 was generated by cloning the A. nidulans pyrG gene from pAlLo2 (WO 2004/099228) into pBM120a. Plasmids pBM120a and pAlLo2 were digested with Nsi I overnight at 37° C. The resulting 4176 bp linearized pBM120a vector fragment and the 1479 bp pyrG gene insert from pAlLo2 were each purified by 0.7% agarose gel electrophoresis using TAE buffer, excised from the gel, and extracted using a QIAQUICK® Gel Extraction Kit.

The 1479 bp pyrG gene insert was ligated to the Nsi I digested pBM120a fragment using a QUICK LIGATION™ Kit (New England Biolabs, Beverly, Mass., USA). The ligation reaction was composed of 1× QUICK LIGATION™ Reaction Buffer (New England Biolabs, Beverly, Mass., USA), 50 ng of Nsi I digested pBM120a vector, 54 ng of the 1479 bp Nsi I digested pyrG gene insert, and 1 μl of T4 DNA Ligase in a total volume of 20 μl. The ligation mixture was incubated at 37° C. for 15 minutes followed at 50° C. for 15 minutes and then placed on ice.

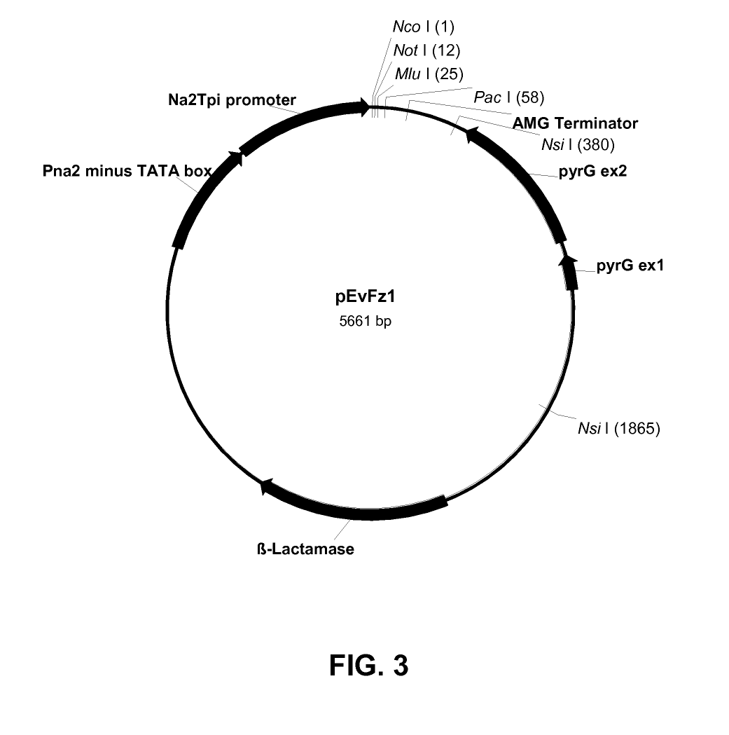

One μl of the ligation mixture was transformed into ONE SHOT® TOP10 chemically competent Escherichia coli cells. Transformants were selected on 2XYT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pEvFz1 (FIG. 3).

### Example 4

### Construction of the Plasmid pDLHD0006 as a Yeast/E. coli/A. oryzae Shuttle Vector

Plasmid pDLHD0006 was constructed as a base vector to enable A. oryzae expression cassette library building using yeast recombinational cloning. Plasmid pDLHD0006 was generated by combining three DNA fragments using yeast recombinational cloning: Fragment 1 containing the E. coli pUC origin of replication, E. coli beta-lactamase (ampR) selectable marker, URA3 yeast selectable marker, and yeast 2 micron origin of replication from pMMar27 (Example 2); Fragment 2 containing the 10 amyR/NA2-tpi promoter (a hybrid of the promoters from the genes encoding Aspergillus niger neutral alpha-amylase and Aspergillus oryzae triose phosphate isomerase and including 10 repeated binding sites for the Aspergillus oryzae amyR transcription factor), Thermomyces lanuginosus lipase open reading frame (ORF), and Aspergillus niger glucoamylase terminator from pJaL1262 (WO 2013/178674); and Fragment 3 containing the Aspergillus nidulans pyrG selection marker from pEvFz1 (Example 3).

| | | PCR |
|---|---|---|
| pDLHD0006 | PCR Contents | Template |
| Fragment 1 | E. coli ori/AmpR/URA/2 micron (4.1 kb) | pMMar27 |
| Fragment 2 | 10 amyR/NA2-tpi PR/lipase/Tamg (4.5 kb) | pJaL1262 |
| Fragment 3 | pyrG gene from pEvFz1 (1.7 kb) | pEvFz1 |

Fragment 1 was amplified using primers 613017 (sense) and 613018 (antisense) shown below. Primer 613017 was designed to contain a flanking region with sequence homology to Fragment 3 (lower case) and primer 613018 was designed to contain a flanking region with sequence homology to Fragment 2 (lower case) to enable yeast recombinational cloning between the three PCR fragments.

Primer 613017 (sense):

(SEQ ID NO: 13)

ttaatcgccttgcagcacaCCGCTTCCTCGCTCACTGACTC
613018 (antisense):

(SEQ ID NO: 14)

acaataaccctgataaatgcGGAACAACACTCAACCCTATCTCGGTC

Fragment 1 was amplified by PCR in a reaction composed of 10 ng of plasmid pMMar27, 0.5 μl of PHUSION® DNA Polymerase (New England Biolabs, Inc., Ipswich, Mass., USA), 20 pmol of primer 613017, 20 pmol of primer 613018, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 1.5 minutes. The resulting 4.1 kb PCR product (Fragment 1) was used directly for yeast recombination with Fragments 2 and 3 below.

Fragment 2 was amplified using primers 613019 (sense) and 613020 (antisense) shown below. Primer 613019 was designed to contain a flanking region of sequence homology to Fragment 1 (lower case) and primer 613020 was designed to contain a flanking region of sequence homology to Fragment 3 (lower case) to enable yeast recombinational cloning between the three PCR fragments.

613019 (sense):

(SEQ ID NO: 15)

agatagggttgagtgttgttccGCATTTATCAGGGTTATTGTCTCATGA
GCGG
613020 (antisense):

(SEQ ID NO: 16)

ttctacacgaaggaaagagGAGGAGAGAGTTGAACCTGGACG

Fragment 2 was amplified by PCR in a reaction composed of 10 ng of plasmid pJaL1262, 0.5 μl of PHUSION® DNA Polymerase, 20 pmol of primer 613019, 20 pmol of primer 613020, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 4.5 kb PCR product (Fragment 2) was used directly for yeast recombination with Fragment 1 above and Fragment 3 below.

Fragment 3 was amplified using primers 613022 (sense) and 613021 (antisense) shown below. Primer 613021 was designed to contain a flanking region of sequence homology to Fragment 2 (lower case) and primer 613022 was designed to contain a flanking region of sequence homology to Fragment 1 (lower case) to enable yeast recombinational cloning between the three PCR fragments.

613022 (sense):

(SEQ ID NO: 17)

aggttcaactctctcctcCTCTTTCCTTCGTGTAGAAGACCAGACAG
613021 (antisense):

(SEQ ID NO: 18)

tcagtgagcgaggaagcggTGTGCTGCAAGGCGATTAAGTTGG

Fragment 3 was amplified by PCR in a reaction composed of 10 ng of plasmid pEvFz1 (Example 3), 0.5 μl of PHUSION® DNA Polymerase, 20 pmol of primer 613021, 20 pmol of primer 613022, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 1.7 kb PCR product (Fragment 3) was used directly for yeast recombination with Fragments 1 and 2 above.

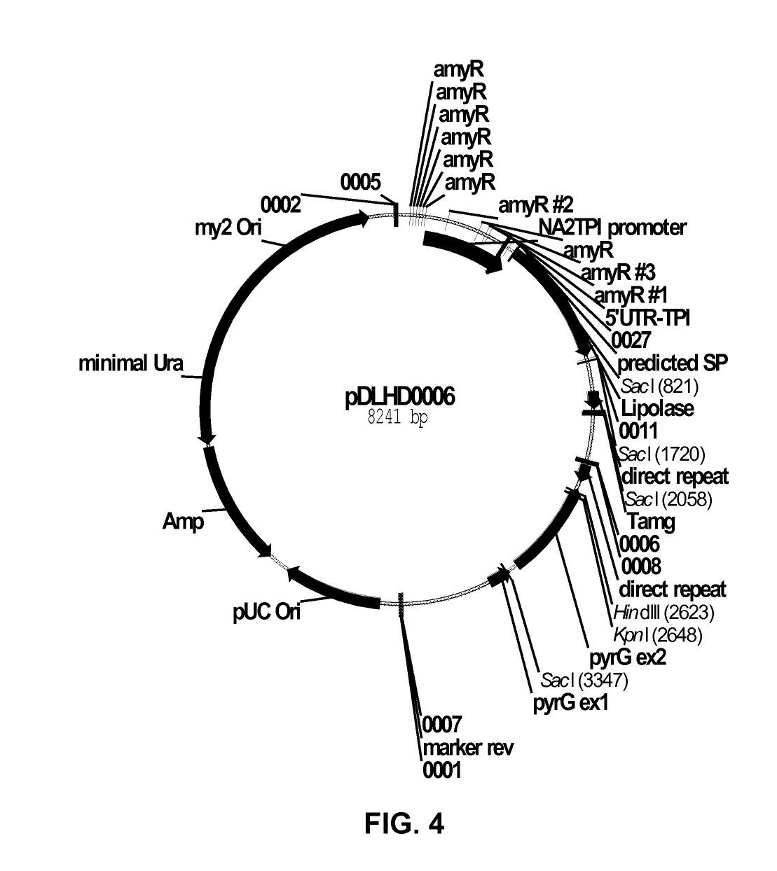

The following procedure was used to combine the three PCR fragments using yeast homology-based recombinational cloning. A 20 μl aliquot of each of the three PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic Defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit. A 3 μl aliquot of the plasmid DNA was then transformed into E. coli ONE SHOT® TOP10 electrocompetent cells according the manufacturer's instructions. Fifty μl of transformed cells were spread onto LB plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated plasmid pDLHD0006 (FIG. 4).

### Example 5

### Preparation of Arabidopsis thaliana Xyloglucan Endotransglycosylase 14

Arabidopsis thaliana xyloglucan endotransglycosylase (AtXET14; SEQ ID NO: 4 [native DNA sequence], SEQ ID NO: 5 [synthetic DNA sequence] and SEQ ID NO: 6 [deduced amino acid sequence]) was recombinantly produced in Aspergillus oryzae JaL355 (WO 2008/138835).

The vector pDLHD0039 was constructed to express the AtXET14 gene in multi-copy in Aspergillus oryzae. Plasmid pDLHD0039 was generated by combining two DNA fragments using restriction-free cloning: fragment 1 containing the AtXET14 ORF and flanking sequences with homology to vector pDLHD0006 (Example 4), and fragment 2 consisting of an inverse PCR amplicon of vector pDLHD0006.

Fragment 1 was amplified using primers AtXET14F (sense) and AtXET14R (antisense) shown below. These primers were designed to contain flanking regions of sequence homology to vector pDLHD0006 (lower case) for ligation-free cloning between the PCR fragments.

Primer AtXET14F (sense):

(SEQ ID NO: 19)

ttcctcaatcctctatatacacaactggccATGGCCTGTTTCGCAACCAA
ACAG
AtXET14R (antisense):

(SEQ ID NO: 20)

agctcgctagagtcgacctaGAGTTTACATTCCTTGGGGAGACCCTG

Fragment 1 was amplified by PCR in a reaction composed of 10 ng of a GENEART® vector pMA containing the AtXET14 synthetic gene SEQ ID NO: 5 [synthetic DNA sequence] cloned between the Sac I and Kpn I sites, 0.5 μl of PHUSION® DNA Polymerase (New England Biolabs, Inc., Ipswich, Mass., USA), 20 pmol of primer AtXET14F, 20 pmol of primer AtXET14R, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 30 seconds. The resulting 0.9 kb PCR product (fragment 1) was treated with 1 μl of Dpn I to remove plasmid template DNA. The Dpn I was added directly to the PCR reaction tube, mixed well, and incubated at 37° C. for 60 minutes, and then column-purified using a MINELUTE® PCR Purification Kit.

Fragment 2 was amplified using primers 614604 (sense) and 613247 (antisense) shown below.

614604 (sense):

(SEQ ID NO: 21)

taggtcgactctagcgagctcgagatc
613247 (antisense):

(SEQ ID NO: 22)

catggccagttgtgtatatagaggattgaggaaggaagag

Fragment 2 was amplified by PCR in a reaction composed of 10 ng of plasmid pDLHD0006, 0.5 μl of PHUSION® DNA Polymerase, 20 pmol of primer 614604, 20 pmol of primer 613247, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 7.3 kb PCR product (fragment 2) was treated with 1 μl of Dpn I to remove plasmid template DNA. Dpn I was added directly to the PCR reaction tube, mixed well, and incubated at 37° C. for 60 minutes, and then column-purified using a MINELUTE® PCR Purification Kit.

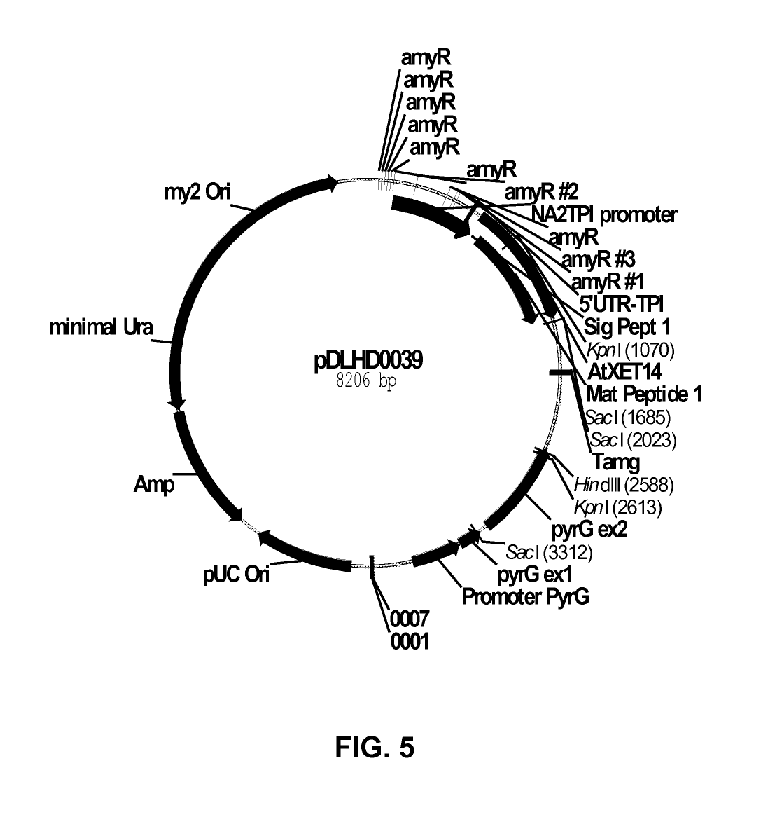

The two PCR fragments were combined using a GENEART® Seamless Cloning and Assembly Kit (Invitrogen, Carlsbad, Calif., USA) according to manufacturer's instructions. Three μl of the resulting reaction product DNA was then transformed into E. coli ONE SHOT® TOP10 electrocompetent cells. Fifty μl of transformed cells were spread onto LB plates supplemented with 100 μg of ampicillin per ml and incubated at 37° C. overnight. Individual transformants were picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit according to the manufacturer's instructions. DNA Sequencing with a 3130XL Genetic Analyzer was used to confirm the presence of each of both fragments in the final plasmid pDLHD0039 (FIG. 5).

Aspergillus oryzae strain JaL355 was transformed with plasmid pDLHD0039 comprising the AtXET14 gene according to the following protocol. Approximately 2-5 x 10⁷ spores of Aspergillus oryzae JaL355 were inoculated into 100 ml of YP+2% glucose+10 mM uridine in a 500 ml shake flask and incubated at 28° C. and 110 rpm overnight. Ten ml of the overnight culture was filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCI-20 mM CaCl₂ and transferred to a sterile 125 ml shake flask containing 20 mg of GLUCANEX® 200 G per ml and 0.2 mg of chitinase per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® into a sterile 50 ml plastic centrifuge tube to remove mycelial debris. The debris in the MIRACLOTH® was washed thoroughly with 0.7 M KCl-20 mM CaCl₂, and centrifuged at 2500 rpm (537×g) for 10 minutes at 20-23° C. The supernatant was removed and the protoplast pellet was resuspended in 20 ml of 1 M sorbitol-10 mM Tris-HCI (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0039 were added to the bottom of a sterile 2 ml plastic centrifuge tube. Then 100 μl of protoplasts were added to the tube followed by 300 μl of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm Minimal medium agar plates. Transformation plates were incubated at 34° C. until transformants appeared.

Thirty-five transformants were picked to fresh Minimal medium agar plates and cultivated at 34° C. for four days until the strains sporulated. Fresh spores were transferred to 48-well deep-well plates containing 2 ml of YP+2% maltodextrin, covered with a breathable seal, and grown for 4 days at 28° C. with no shaking. After 4 days growth the culture medium was assayed for xyloglucan endotransglycosylase activity using an iodine stain assay, and for xyloglucan endotransglycosylase expression by SDS-PAGE.

Xyloglucan endotransglycosylase activity was measured using the iodine stain assay described in Example 1. The assay demonstrated the presence of xyloglucan endotransglycosylase activity in several transformants.

SDS-PAGE was performed as described in Example 1. SDS-PAGE revealed a band of approximately 32 kDa corresponding to AtXET14 in several transformants.

### Example 6

### Purification of Vigna angularis Xyloglucan Endotransglycosylase 16

One liter solutions of crude fermentation broth of Vigna angularis were filtered using a 0.22 μm STERICUP® filter (Millipore, Bedford, Mass., USA) and the filtrates were stored at 4° C. Cell debris was resuspended in 1 liter of 0.25% TRITON® X-100 (4-(1,1,3,3-tetramethylbutyl)phenyl-polyethylene glycol; Sigma Aldrich, St. Louis, Mo., USA)-20 mM sodium citrate pH 5.5, incubated at least 30 minutes at room temperature, and then filtered using a 0.22 μm STERICUP® filter. The filtrates containing VaXET16 were pooled and concentrated to a volume between 500 and 1500 ml using a VIVAFLOW® 200 tangential flow concentrator (Millipore, Bedford, Mass., USA) equipped with a 10 kDa molecular weight cutoff membrane.

The concentrated filtrates were loaded onto a 150 ml Q SEPHAROSE® Big Beads column (GE Healthcare Lifesciences, Piscataway, N.J., USA), pre-equilibrated with 20 mM sodium citrate pH 5.5, and eluted isocratically with the same buffer. The eluent was loaded onto a 75 ml Phenyl SEPHAROSE® HP column (GE Healthcare Lifesciences, Piscataway, N.J., USA) pre-equilibrated in 20% ethylene glycol-20 mM sodium citrate pH 5.5. VaXET16 was eluted using a linear gradient from 20% to 50% of 70% ethylene glycol in 20 mM sodium citrate pH 5.5 over 4 column volumes.

Purified VaXET16 was quantified using a BCA assay (Pierce, Rockford, Ill., USA) in 96-well plate format with bovine serum albumin (Pierce, Rockford, Ill., USA) as a protein standard at concentrations between 0 and 2 mg/ml and was determined to be 1.40 mg/ml. VaXET16 homogeneity was confirmed by the presence of a single band of approximately 32 kDa using a 8-16% gradient CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

The activity of the purified VaXET16 was determined by measuring the rate of incorporation of fluorescein isothiocyanate-labeled xyloglucan oligomers into tamarind seed xyloglucan (Megazyme, Bray, UK) by fluorescence polarization (as described in Example 9). The apparent activity was 18.5±1.2 P s⁻¹mg⁻¹.

The purified VaXET16 preparation was tested for background activities xylanase, amylase, cellulase, beta-glucosidase, protease, amyloglucosidase, and lipase using standard assays as shown below.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethyliden- | 4-fold | FAU(F) | ND |
| | G7-pNp | | | |
| Cellulase CNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 740 |
| (EnzCheck) | | | | |
| Protease, pH 9 | Casein | none | KMTU | 332 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.02 |

### Example 7

### Purification of Arabidopsis thaliana Xyloglucan Endotransglycosylase 14

The purification and quantification of the Arabidopsis thaliana xyloglucan endotransglycosylase 14 (AtXET14) was performed as described for VaXET16 in Example 6, except that elution from the Phenyl SEPHAROSE® HP column was performed using a linear gradient from 40% to 90% of 70% ethylene glycol in 20 mM sodium citrate pH 5.5 over 4 column volumes.

AtXET14 homogeneity was confirmed by the presence of a single band of approximately 32 kDa using a 8-16% CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

Purified AtXET14 was quantified using a BCA assay in a 96-well plate format with bovine serum albumin as a protein standard at concentrations between 0 and 2 mg/ml and was determined to be 1.49 mg/ml.

The activity of the purified AtXET14 was determined as described in Example 9. The apparent activity was 34.7±0.9 P s⁻¹mg⁻¹.

The purified AtXET14 preparation was tested for background activities including xylanase, amylase, cellulase, beta-glucosidase, protease, amyloglucosidase, and lipase using standard assays as shown below.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethyliden- | 4-fold | FAU(F) | ND |
| | G7-pNp | | | |
| Cellulase GNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 82 |
| (EnzCheck) | | | | |
| Protease, pH 9 | Casein | none | KMTU | 53 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.24 |

### Example 8

### Generation of Fluorescein Isothiocyanate-Labeled Xyloglucan

Fluorescein isothiocyanate-labeled xyloglucan oligomers (FITC-XGOs) were generated by reductive amination of the reducing ends of xyloglucan oligomers according to the procedure described by Zhou et al., 2006, Biocatalysis and Biotransformation 24: 107-120), followed by conjugation of the amino groups of the XGOs to fluorescein isothiocyanate isomer I (Sigma Aldrich, St. Louis, Mo., USA) in 100 mM sodium bicarbonate pH 9.0 for 24 hours at room temperature. Conjugation reaction products were concentrated to dryness in vacuo, dissolved in 0.5 ml of deionized water, and purified by silica gel chromatography, eluting with a gradient from 100:0:0.04 to 70:30:1 acetonitrile:water:acetic acid as mobile phase. Purity and product identity were confirmed by evaporating the buffer, dissolving in D₂O (Sigma Aldrich, St. Louis, Mo., USA), and analysis by ¹H NMR using a Varian 400 MHz MercuryVx (Agilent, Santa Clara, Calif., USA). Dried FITC-XGOs were stored at −20° C. in the dark, and were desiccated during thaw.

Twenty-four ml of 10 mg of tamarind seed xyloglucan (Megazyme, Bray, UK) per ml of deionized water, 217 μl of 7.9 mg of FITC-XGOs per ml of deionized water, 1.2 ml of 400 mM sodium citrate pH 5.5, and 600 μl of 1.4 mg of VaXET16 per ml of 20 mM sodium citrate pH 5.5 were mixed thoroughly and incubated overnight at room temperature. Following overnight incubation, FITC-XG was precipitated by addition of ice cold ethanol to a final volume of 110 ml, mixed thoroughly, and incubated at 4° C. overnight. The precipitated FITC-XG was washed with water and then transferred to Erlenmeyer bulbs. Residual water and ethanol were removed by evaporation using an EZ-2 Elite evaporator (SP Scientific/Genevac, Stone Ridge, N.Y., USA) for 4 hours. Dried samples were dissolved in water, and the volume was adjusted to 48 ml with deionized water to generate a final FITC-XG concentration of 5 mg per ml with an expected average molecular weight of 100 kDa.

### Example 9

### Fluorescence Polarization Assay for Xyloglucan Endotransglycosylation Activity

Xyloglucan endotransglycosylation activity was assessed using the following assay. Reactions of 200 μl containing 1 mg of tamarind seed xyloglucan per ml, 0.01 mg/ml FITC-XGOs prepared as described in Example 8 and 10 μl of appropriately diluted XET were incubated for 10 minutes at 25° C. in 20 mM sodium citrate pH 5.5 in opaque 96-well microtiter plates. Fluorescence polarization was monitored continuously over this time period, using a SPECTRAMAX® M5 microplate reader (Molecular Devices, Sunnyvale, Calif., USA) in top-read orientation with an excitation wavelength of 490 nm, an emission wavelength of 520 nm, a 495 cutoff filter in the excitation path, high precision (100 reads), and medium photomultiplier tube sensitivity. XET-dependent incorporation of fluorescent XGOs into non-fluorescent XG results in increasing fluorescence polarization over time. The slope of the linear regions of the polarization-time progress curves was used to determine the activity.

### Example 10

### Binding of Fluorescein Isothiocyanate-Labeled Xyloglucan to Kaolin

To test and quantify kaolin-binding by xyloglucan, fluorescein isothiocyanate-labeled xyloglucan (FITC-XG) was used as a reporter and residual solution fluorescence was measured following incubation in either the presence or absence of kaolin. FITC-XG was generated as described in Example 8. Vigna angularis XET16 was purified as described in Example 6.

Binding reactions of 500 μl were performed in sealed 1.1 ml 96-deep well plates (Axygen, Union City, Calif., USA). Kaolin (Sigma Aldrich, St. Louis, Mo., USA) in an amount of 0 to 20 mg per ml was incubated with or without 1 μM VaXET16, and with or without 1 mg of FITC-XG per ml of 50 mM sodium citrate pH 5.5 at either 25° C. or 37° C. in an INNOVAO 40 shaker incubator (New Brunswick Scientific, Enfield, Conn., USA) for up to 5 days. Plates were sealed with an ALPS 3000 μlate sealer (Thermo Scientific, Waltham, Mass., USA) and then wrapped in aluminum foil to preserve the fluorophore during incubation.

Following incubation for 1, 2, and 5 days, the deep well plates were centrifuged at 3000 rpm for 5 minutes using a LEGEND™ RT Plus centrifuge (Thermo Scientific, Waltham, Mass., USA) to pellet the kaolin with any associated FITC-XG and fluorescence intensity of the supernatant was measured in the following manner. Aliquots of 200 μl of each supernatant were removed and transferred to a Costar 9017 flat bottomed microtiter plate (Corning, Tewksbury, Mass., USA). Fluorescence intensity was measured using a SPECTRAMAX® M5 microplate reader (Molecular Devices, Sunnyvale, Calif., USA) in bottom read format with an excitation wavelength of 488 nm, emission wavelength of 520 nm, and cutoff filter of 495 nm, in high precision mode (100 reads) and medium photomultiplier tube sensitivity settings. Fluorescence spectra were measured using the same samples and excitation settings as described for intensity measurements, measuring emission at wavelengths from 500 to 625 nm.

After measuring intensity, each sample aliquot was returned to its original reaction. The plate was resealed, rewrapped in foil, and placed back in the incubator to continue the binding reaction.

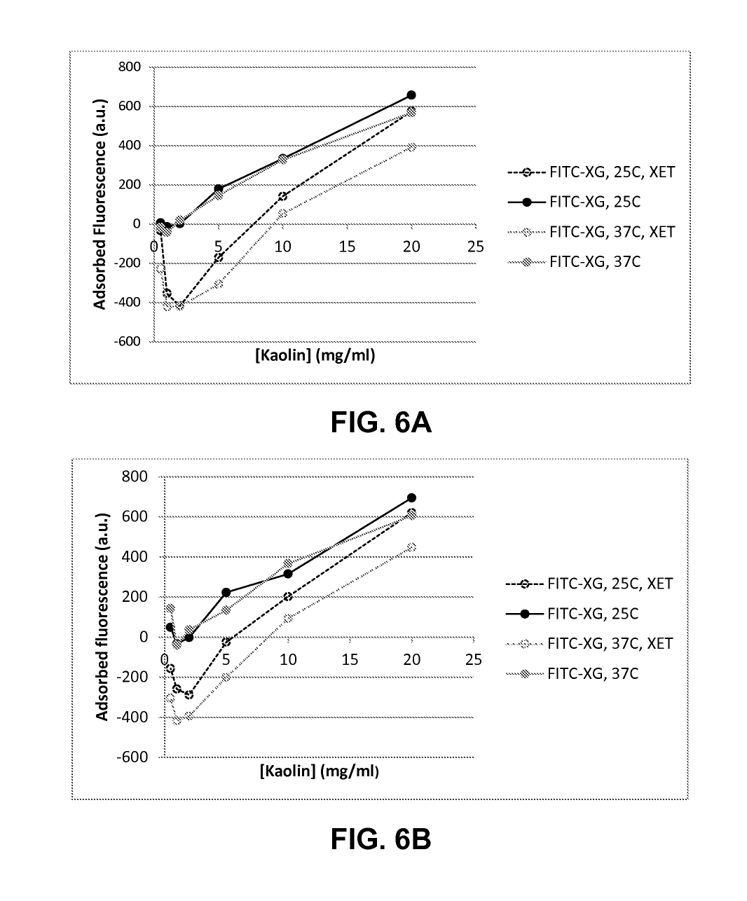

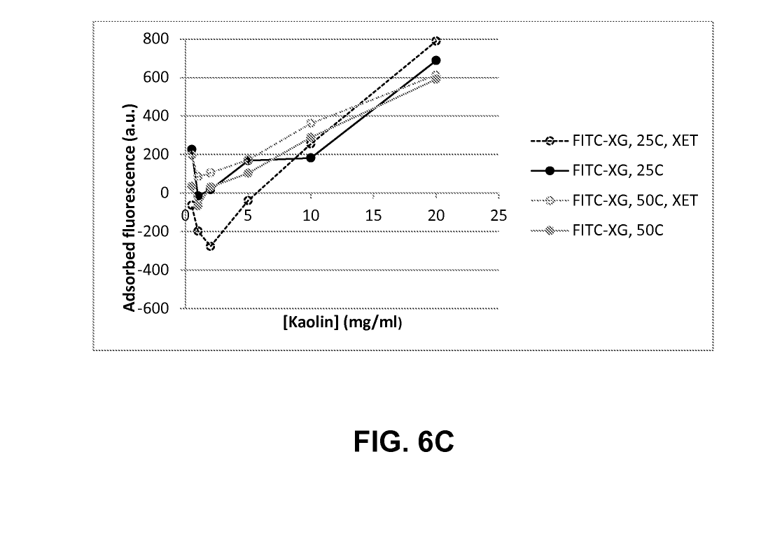

FIG. 6 shows the increase of FITC-XG fluorescence adsorbed to kaolin with increasing mass of kaolin, relative to a control incubation performed without kaolin. FIG. 6A shows kaolin titration after 1 day of incubation; FIG. 6B shows kaolin titration after 2 days of incubation; and FIG. 6C shows kaolin titration after 5 days of incubation. With increasing masses of kaolin, adecreasing fluorescence intensity in the supernatant phase and higher fluorescence adsorbed to the kaolin were observed. Similarly, in the presence of VaXET16, as the amount of kaolin in the reaction increased, the amount of fluorescence associated with the kaolin increased. At very low concentrations of kaolin, the solution phase fluorescence increased rather than decreased, yielding an apparent adsorbed fluorescence of less than zero.

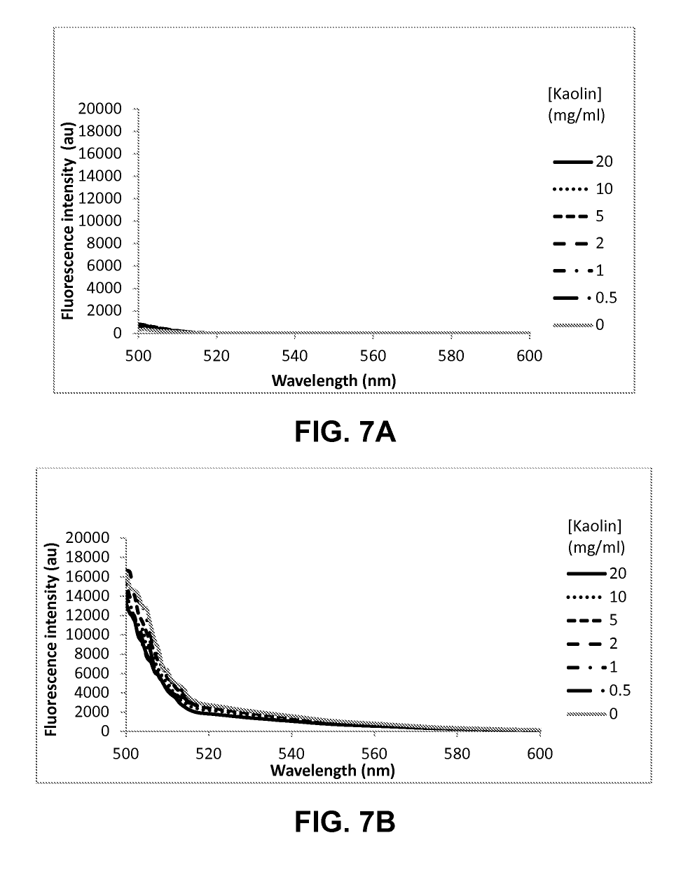

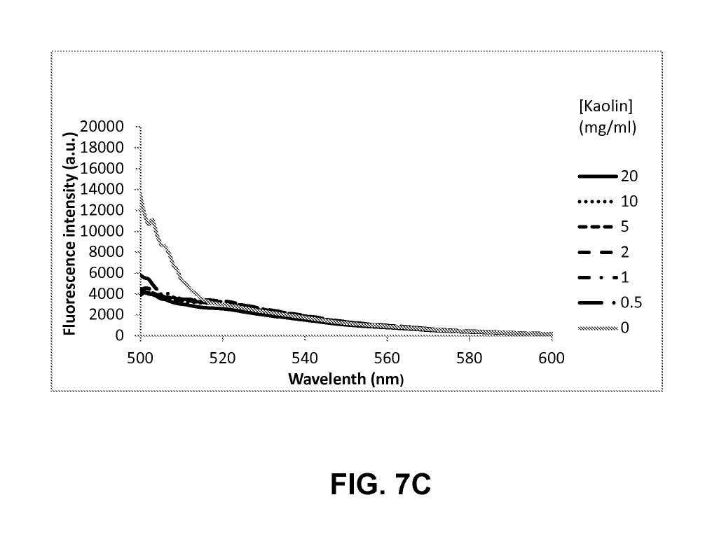

To confirm that fluorescence intensity did increase, fluorescence spectra were measured and are shown in FIG. 7. FIG. 7A shows the fluorescence spectra of supernatants of various kaolin concentrations incubated without FITC-XG. FIG. 7B shows the fluorescence spectra of supernatants of various kaolin concentrations incubated with FITC-XG. FIG. 7C shows the fluorescence spectra of supernatants of various concentrations of kaolin incubated with FITC-XG and VaXET16.

In the absence of VaXET16, high emission intensity was observed from 500 to 520 nm, which was attributed to scatter of the excitation light caused by aggregates of the xyloglucan. In the presence of VaXET16, when kaolin was absent a similar light scatter peak was observed. However when kaolin was present the emission intensity between 500 and 520 nm was dramatically reduced, indicating a sharp reduction in light scatter. These results indicate that the average particle size in solution was much smaller, thus the xyloglucan aggregates were dispersed by VaXET16 in the presence of kaolin. In both cases, the xyloglucan was bound to kaolin, modifying the kaolin with polysaccharide and functionalizing the kaolin with the fluorescent dye. When VaXET16 was present, the xyloglucan appeared more dispersed in the presence of kaolin than when VaXET16 was absent.

### Example 11

### Binding of Fluorescein Isothiocyanate-Labeled Xyloglucan to Kaolin by Confocal Microscopy

The reaction mixtures described in Example 10 were analyzed by laser scanning confocal microscopy according the following procedure. Aliquots of 300 μl of each reaction were removed, transferred to a 96-well, 0.45 micron PVDF filter plate (Millipore, Billerica, Mass., USA), and centrifuged at 3000 rpm for 10 minutes using a LEGEND™ RT Plus centrifuge. The retentates were washed three times by resuspension in 300 μl of deionized water, mixed thoroughly, and then centrifuged as above. Washed kaolin retentates were then resuspended in 300 μl of deionized water and transferred to microcentrifuge tubes. Samples were stored at 4° C. until analyzed. Approximately 20 μl of each sample were applied to a FisherFinest Premium 3″x1″x 1 mm microscope slide (Fisher Scientific, Inc., Pittsburg, Pa., USA) and covered with a Fisherbrand 22×22-1.5 microscope coverslip (Fisher Scientific, Inc., Pittsburg, Pa., USA) before sealing the coverslip to the slide using clear nail polish.

Fluorescence arising from fluorescein isothiocyanate-labeled xyloglucan (FITC-XG) associated with kaolin was imaged using an Olympus FV1000 laser scanning confocal microscope (Olympus, Center Valley, Pa., USA) with a 10× air gap objective lens. Excitation was performed using the 488 nm line of the argon ion laser, and emission intensity was detected by integrating intensity from 500 to 520 nm incident on the photomultiplier tube detector through an emission monochromator. The photomultiplier (PMT) voltage settings were 678 with an offset setting of 3 for all images. Post scan image analysis was performed using FIJI (NIH, Bethesda, Md., USA) and MATLAB® (The Mathworks, Natick, Mass., USA).

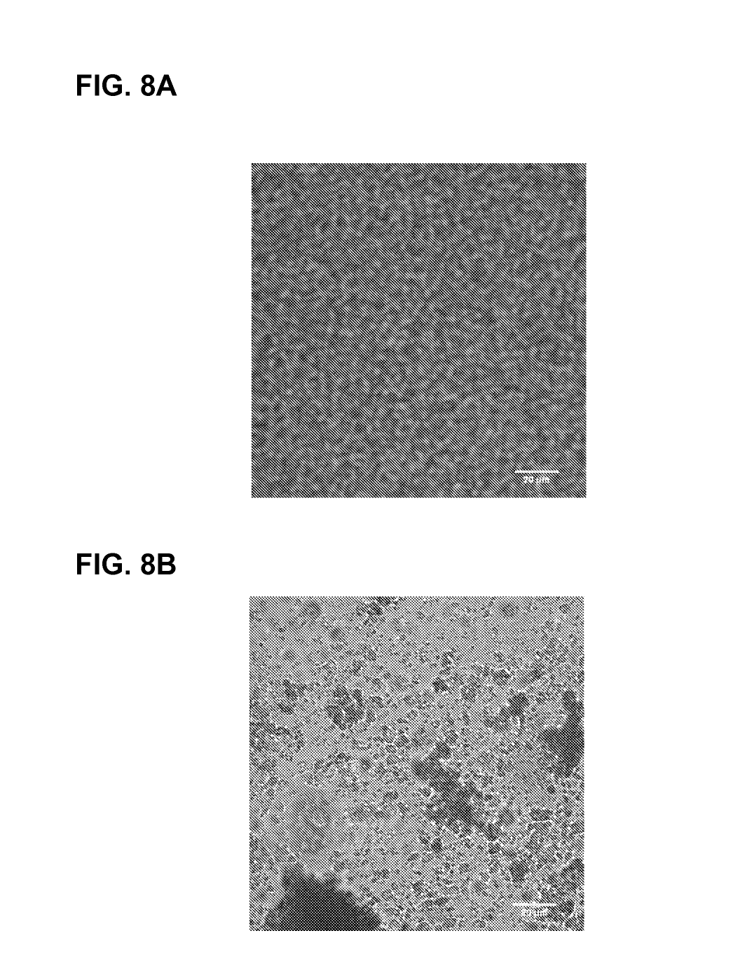

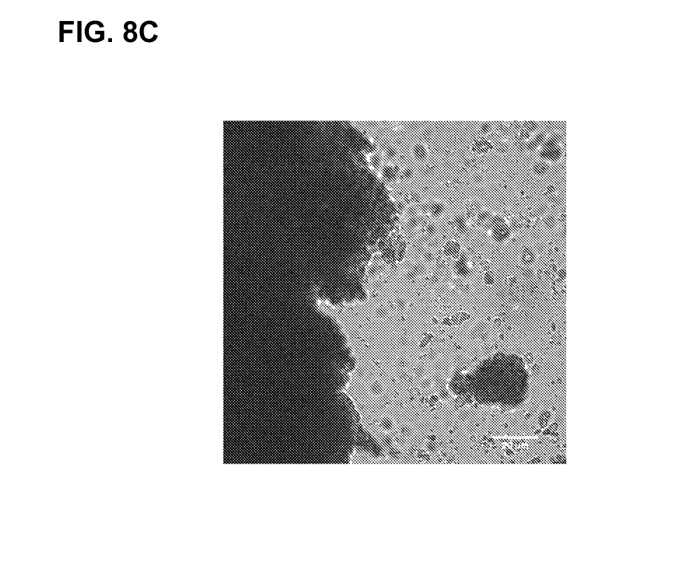

FIG. 8A shows the confocal microscopy image of kaolin incubated with no FITC-XG, overlaying the fluorescence emission (false colored in green) with transmittance on the left, and the threshold filtered emission intensity image on the right. From the image, no substantial fluorescence intensity was observed. The average pixel intensity was 56.69±23.92.

FIG. 8B shows the confocal microscopy image of kaolin incubated with FITC-XG, overlaying the fluorescence emission (false colored in green) with transmittance on the left, and the threshold filtered emission intensity image on the right. The average pixel intensity was 211.49±159.37.

FIG. 8C shows the confocal microscopy image of kaolin incubated with FITC-XG and VaXET16, overlaying the fluorescence emission (false colored in green) with transmittance on the left, and the threshold filtered emission intensity image on the right. The average pixel intensity was 185.26±161.28.

Comparing the 3 images, clear differences were observed. Kaolin incubated without FITC-XG had a fluorescence intensity only slightly above background and significantly less fluorescence intensity than kaolin incubated with FITC-XG or FITC-XG and VaXET16. The rheology of the kaolin was also clearly different between samples incubated with and without FITC-XG. Kaolin was uniformly dispersed and appeared homogenous at this level of magnification when incubated without FITC-XG. Conversely, in the presence of FITC-XG or FITC-XG and VaXET16, the kaolin appeared to cluster or aggregate, and bright fluorescent spots were observed. Since the samples were extensively washed prior to microscopy, the fluorescent spots arose from FITC-XG bound to the kaolin, and these images indicate that FITC-xyloglucan had altered the rheology of kaolin.

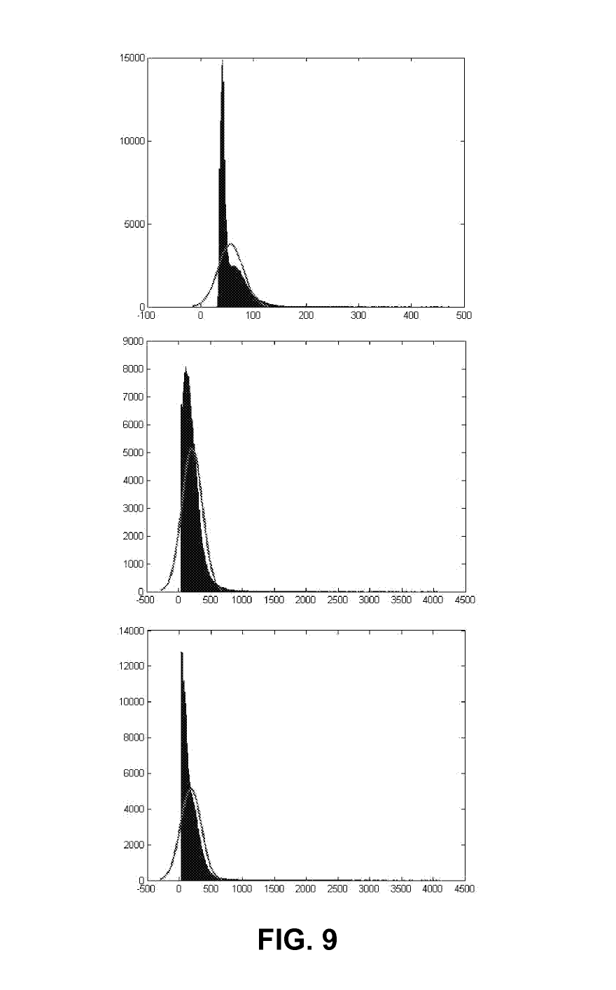

Quantitative analysis of the microscope images was performed to delineate differences between kaolin incubated with FITC-XG and kaolin incubated with FITC-XG and VaXET16. FIG. 9 shows histograms of pixel intensities for the 3 images. FIG. 9A shows a pixel intensity histogram for the kaolin incubated with no FITC-XG. FIG. 9B shows a pixel intensity histogram for the kaolin incubated with FITC-XG. FIG. 9C shows a pixel intensity histogram for the kaolin incubated with FITC-XG and VaXET16. From the intensity histograms, it is clear that almost no intensity was observed from the kaolin incubated with no FITC-XG. Comparing the FITC-XG incubation with the FITC-XG and VaXET16 incubation, a broader distribution of intensities, a higher mean intensity, and higher frequency of pixels with a higher number of counts were observed for kaolin incubated with FITC-XG. The images were threshold-filtered to remove background fluorescence, and the diameters of the remaining fluorescent spots were determined. Comparing the histograms of the spot sizes, the image of kaolin incubated with FITC-XG and VaXET16 showed a higher frequency of smaller fluorescent particles relative to the FITC-XG incubated kaolin. These data indicate that VaXET16 functioned to reduce the size of the xyloglucan particles associated with kaolin, generating more and smaller FITC-XG spots. The data is consistent with the reduction of light scattering observed in the solution fluorescence experiments performed in Example 10, and confirmed a role for VaXET16 in altering the properties of kaolin-FITC-XG material.

### Example 12

### Changes in Kaolin Physical Properties after Incubation with Xyloglucan or Xyloglucan and Vigna angularis Xyloglucan Endotransglycosylase 16

In 100 ml glass bottles, 5 grams of kaolin (Sigma Aldrich, St. Louis, Mo., USA) were incubated with or without 2.375 mg/ml tamarind seed xyloglucan, with or without 1.1 μM VaXET16 in 20 mM sodium citrate pH 5.5. The samples were mixed thoroughly, then placed horizontally in an INNOVA® 40 shaker incubator and incubated at 25° C. overnight with shaking at 150 rpm. After incubation, the samples were mixed vigorously and then aliquoted into two 50 ml Centristar conical tubes (Corning, Tewksbury, Mass., USA). The aliquots were centrifuged at 3200 rpm for 40 minutes using a LEGEND™ RT Plus centrifuge. The kaolin pellets were either resuspended and stored at 4° C. or the supernatants were decanted and the kaolin pellets resuspended in approximately 50 ml of deionized water. The resuspended samples were incubated at 25° C. overnight with shaking at 150 rpm, centrifuged, decanted, resuspended in 50 ml of deionized water, and incubated overnight with shaking at 150 rpm two additional times, for a total of 3 washes.

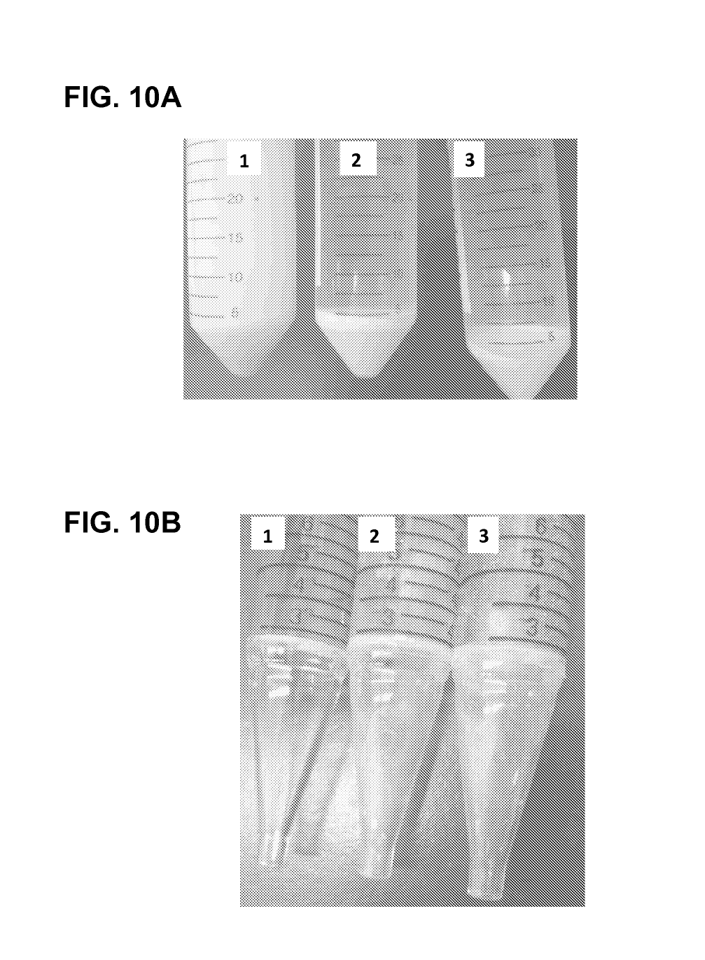

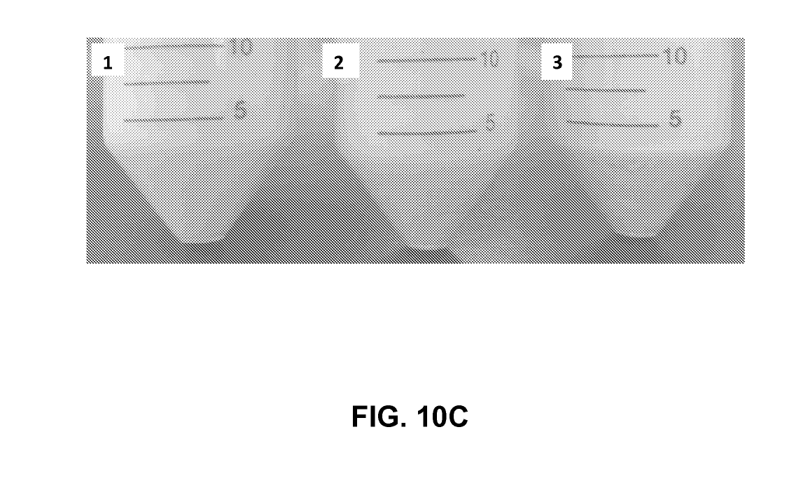

FIG. 10A shows photographs of the 50 ml conical tubes containing (1) kaolin, (2) kaolin incubated with xyloglucan, and (3) kaolin incubated with xyloglucan and VaXET16, following centrifugation. Kaolin treated with xyloglucan or xyloglucan and VaXET16 completely pelleted out during centrifugation while untreated kaolin remained partially suspended. These results indicate that the mass of the kaolin particles treated with xyloglucan increased or their density decreased; both are indications that xyloglucan associated with the kaolin, and possibly facilitated binding of kaolin particles together.

FIG. 10B shows photographs of polystyrene serological pipets following contact with (1) kaolin, (2) kaolin incubated with xyloglucan, and (3) kaolin incubated with xyloglucan and VaXET16. Kaolin treated with xyloglucan or with xyloglucan and VaXET16 adhered to the polystyrene. These results indicate that xyloglucan facilitated binding of kaolin particles to plastic.

FIG. 10C shows photographs of the 50 ml conical tubes containing (1) kaolin, (2) kaolin incubated with xyloglucan, and (3) kaolin incubated with xyloglucan and VaXET16, following extensive washing and resuspension in water. Kaolin treated with xyloglucan or with xyloglucan and VaXET16 and then extensively washed and centrifuged did not fully resuspend when applied to a vortex mixer. Large particles of kaolin appeared to clump or aggregate together and settled quickly. These results indicate that, even following extensive washing, xyloglucan remained bound to kaolin particles and when the particles were compressed by centrifugation, xyloglucan bound the kaolin particles together. These observations were consistent with the confocal microscopy data described in Example 11; kaolin clustered or aggregated when incubated with xyloglucan or xyloglucan and VaXET16. Additionally, the data indicates that incubation of kaolin with xyloglucan or xyloglucan and VaXET16 increased the adhesion of kaolin to surfaces or other substances such as polystyrene.

### Example 13

### Effect of Vigna angularis Xyloglucan Endotransglycosylase 16 Modified Kaolin on Filler Retention in Handsheet Compositions

A 0.3% (w/w) slurry of bleached eucalyptus kraft fiber (BEKP) was prepared with tap water. To prepare a single hand sheet, 800 ml of the slurry, containing 2.4 oven dry grams of fiber, were transferred to a 1 liter plastic beaker. Aliquots of water (control) or kaolin slurry were added to the blender. The kaolin slurries were generated by suspending 2 g of kaolin (Sigma Aldrich, St. Louis, Mo., USA) in 50 ml of deionized water, or in 50 ml of 20 mM citrate pH 5.5, or in 50 ml of 20 mM citrate pH 5.5 containing 125 mg of tamarind kernel powder xyloglucan with or without 1 μM VaXET16. Unmodified and modified kaolin slurries were dosed to deliver a 10% equivalent weight with the fiber in the sample (i.e., 0.24 oven dry grams of kaolin per sample). The samples were then mixed for 30 seconds by an impeller at low speed. Immediately after mixing, each sample was transferred to the half-full deckle of a standard hand sheet former. The deckle was then completely filled, agitated, and drained to form a sheet. Each sheet was couched, pressed, and dried according to TAPPI standard procedure T-205 sp-95 “Forming Handsheets for Physical Testing of Pulp”. Eight sheets were prepared from each of the trial sets listed in Table 1.

Three sheets from each set were used to determine ash content according to TAPPI standard T-211 om-02 “Ash in Wood, Pulp, Paper and Paperboard: Combustion at 525° C”. The physical strength properties of the remaining five sheets from each set were determined according to TAPPI standard T-220 sp-96 “Physical Testing of Pulp Handsheets”.

TABLE 1
Trial description

| | | Total fiber | Kaolin/Fiber | | # of |
|---|---|---|---|---|---|
| Trial | Fiber type | (odg) | % (w/w) | Clay description | sheets |
| 1 | BEKP | 2.4 | 0 | None | 8 |
| 2 | BEKP | 2.4 | 10 | Kaolin in water | 8 |
| 3 | BEKP | 2.4 | 10 | Kaolin in citrate | 8 |
| | | | | buffer | |
| 4 | BEKP | 2.4 | 10 | Kaolin + XG | 8 |
| 5 | BEKP | 2.4 | 10 | Kaolin + XG + | 8 |
| | | | | VaXET16 | |
| 6 | BEKP | 2.4 | 10 | Kaolin + | 8 |
| | | | | VaXET16 | |

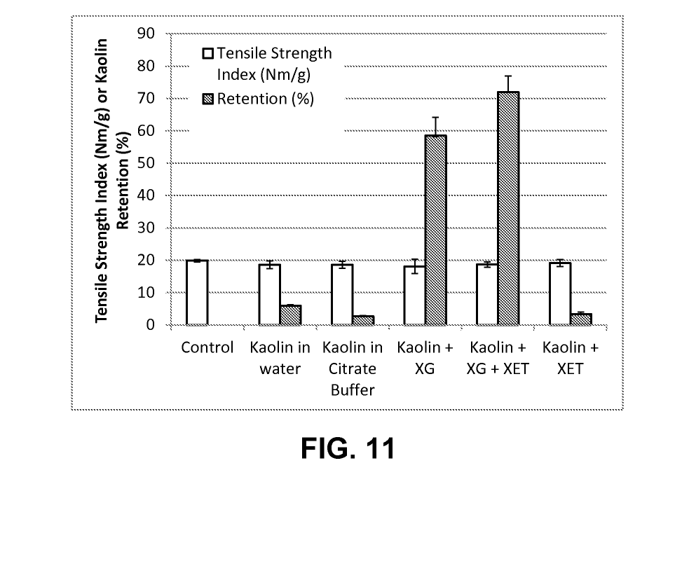

FIG. 11 shows the filler retention results. Although interactions between kaolin and xyloglucan resulted in significant retention of the filler in the forming web of BEKP, modification of the xylogucan by VaXET16 in the presence of kaolin produced a material with even greater retention. The results suggest that modification of kaolin in the presence of XET and xyloglucan produced filler with significantly improved retention in fibrous webs (e.g., paper and board).

Increased retention of mineral fillers is desirable to reduce costs and impart specific optical and performance properties of paper and board. However, minerals obstruct the fiber-to-fiber bonding that is essential to build strength in the final product. Therefore, an upper limit of inclusion generally exists. The physical testing results indicated that, even with the significantly improved retention of kaolin, the strength properties of the handsheets were not significantly affected (FIG. 11).

Table 2 lists the physical properties of the handsheets, tested as described above. For most of the properties, the change is small despite the large increase in kaolin retained in the handsheets when incubated with xyloglucan and VaXET16. These data indicate that xyloglucan and particularly xyloglucan with VaXET16 can be used to increase the retention of kaolin filler in paper without the use of flocculants or other retention aids, while maintaining the physical properties of the paper produced.

TABLE 2
Physical testing and ash content data obtained from 120 g/m² handsheets prepared
from bleached eucalyptus fiber in the absence or presence of various kaolin slurries.

| | | | | | | Kaolin + | |
|---|---|---|---|---|---|---|---|
| Physical | | | Kaolin in | Kaolin in | Kaolin + | XG + | Kaolin + |
| Test | Units | Control | water^{¶} | citrate^{¶} | XG^{¶} | XET^{¶} | XET^{¶} |
| Basis wt. | g/m² | 120.8 | 122.1 | 121.4 | 127.8 | 131.0 | 122.3 |
| Caliper | Mils | 0.05 | 0.05 | 0.05 | 0.05 | 0.05 | 0.05 |
| App. | g/cm³ | 2.5 | 2.6 | 2.6 | 2.6 | 2.6 | 2.6 |
| Density | | | | | | | |
| Dry | (Max | 36.0 | 34.0 | 33.8 | 34.7 | 36.7 | 35.1 |
| Tensile | load) | | | | | | |
| | kN/m | | | | | | |
| Bulk | cm³/g | 0.40 | 0.39 | 0.39 | 0.39 | 0.38 | 0.39 |
| Elongation | % | 2.6 | 2.5 | 2.6 | 2.4 | 2.7 | 2.7 |
| TEA† | J/m² | 49.6 | 43.4 | 43.7 | 41.2 | 50.1 | 46.5 |
| TI‡ | N · m/g | 19.9 | 18.6 | 18.6 | 18.1 | 18.7 | 19.1 |
| Burst | kPa · m²/g | 1.5 | 1.5 | 1.5 | 1.4 | 1.4 | 1.5 |
| Index | | | | | | | |
| Tear | mN · m²/g | 7.8 | 5.9 | 6.8 | 8.2 | 6.9 | 6.6 |
| Index | | | | | | | |
| Ash | % | 0.4 | 0.1 | 0.6 | 5.9 | 6.9 | 0.7 |
| Content | | | | | | | |

†TEA refers to Tensile Energy Absorption.
‡TI refers to Tensile Index.
^{¶}Kaolin was added to the slurry before handsheet formation at 10% (w/w) of the dry fiber.

### Example 14

### Binding of Fluorescein Isothiocyanate-Labeled Xyloglucan Binding to Titanium (IV) Oxide

To test and quantify titanium (IV) oxide-binding by xyloglucan, fluorescein isothiocyanate-labeled xyloglucan (FITC-XG) was used as a reporter and residual solution fluorescence was measured following incubation in either the presence or absence of titanium (IV) oxide (TiO₂). FITC-XG was generated as described in Example 8. Arabidopsis thaliana XET14 (AtXET14) was purified as described in Example 7. Binding was assessed as described in Example 10, with the following exceptions. A 10% slurry was generated by suspending 1 g of TiO₂ (mixture of rutile and anatase, particle size<100 nm) (Sigma Aldrich, St. Louis, Mo., USA) in 10 ml of 20 mM sodium citrate pH 5.5. The slurry was resuspended by inversion and 200 μl were pipetted to each binding reaction. TiO₂-binding reactions of 500 μl in 20 mM sodium citrate pH 5.5 contained TiO₂ and either 1 mg/ml FITC-XG or 1 mg/ml FITC-XG with 1 μM AtXET14. Control reactions contained TiO₂ with no FITC-XG and AtXET14, or FITC-XG without TiO₂. Samples were mixed thoroughly with a pipet and then incubated under ambient conditions for 48 hours. At the indicated times, the 1.1 ml, 96-deep well plates were centrifuged at 3000 rpm (˜2200×g) for 15 minutes, 100 μl aliquots of each of the supernatants were removed, and fluorescence intensity measured as described in Example 4. Aliquots were returned to their respective reaction well, wells were mixed thoroughly with a pipet, and the plates were resealed.

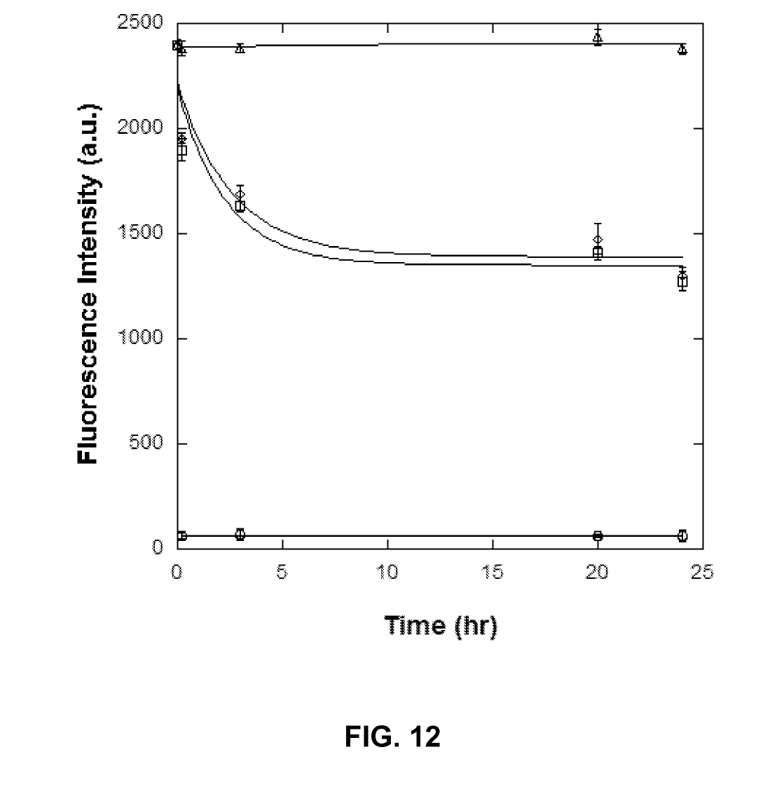

FIG. 12 shows the fluorescence intensity of the supernatants of TiO₂-binding reactions and control incubations at various times. Open circles: TiO₂ with no FITC-XG; squares: TiO₂ with FITC-XG; diamonds: TiO₂ with FITC-XG and AtXET14; triangles: FITC-XG with no TiO₂. From the plot, it is evident that the fluorescence intensity of the supernatant of TiO2 incubated with FITC-XG or FITC-XG and AtXET14 decreased sharply with time as the FITC-XG bound to TiO₂ and was removed from solution. Conversely, FITC-XG fluorescence did not decrease, and TiO₂ had a fluorescence intensity indistinguishable from the background. These data indicate that FITC-XG bound to TiO₂.

### Example 15

### Changes in Titanium (IV) Oxide Physical Properties after Incubation with Xyloglucan or Xyloglucan and Arabidopsis thaliana Xyloglucan Endotransglycosylase 14.

Titanium (IV) oxide binding reactions were prepared as described in Example 14. Immediately following the initial mixing, the 1.1 ml, 96-deep well plates were centrifuged at 3000 rpm (approximately 2200×g) for 1 minute.

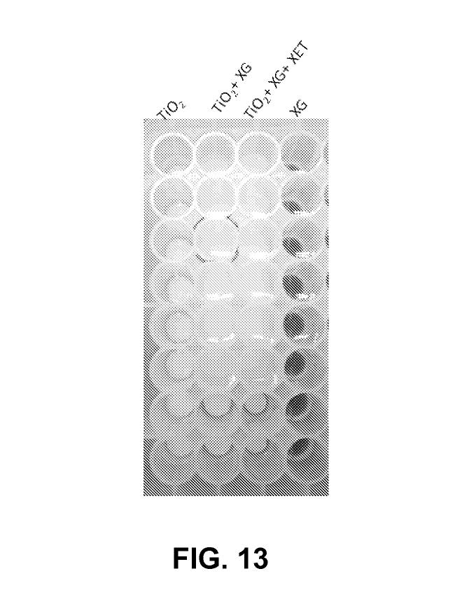

FIG. 13 shows a photograph of the TiO₂ suspensions taken immediately after centrifugation. From the figure it is clear that TiO2 treated with xyloglucan or xyloglucan and AtXET14 had a greater sedimentation coefficient, or were opposed by a lower buoyant force indicating that the density of the TiO₂ particles had decreased or the mass had increased. As with kaolin discussed in Example 12, xyloglucan had therefore associated with TiO₂ and potentially crosslinked TiO₂ particles together.

The inventions described and claimed herein are not to be limited in scope by the specific aspects herein disclosed, since these aspects are intended as illustrations of several aspects of the invention. Any equivalent aspects are intended to be within the scope of the inventions. Indeed, various modifications of the inventions in addition to those shown and described herein will become apparent to those skilled in the art from the foregoing description. Such modifications are also intended to fall within the scope of the appended claims. In the case of conflict, the present disclosure including definitions will control.

1. A process for modifying a filler material comprising treating a suspension of the filler material with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase, under conditions leading to a modified filler material, wherein the modified filler material possesses an improved property compared to the unmodified filler material.

2. The process of claim 1, wherein the average molecular weight of the polymeric xyloglucan ranges from 2 kDa to about 500 kDa.

3. The process of claim 1 or 2, wherein the average molecular weight of the xyloglucan oligomer ranges from 0.5 kDa to about 500 kDa.

4. The process of any of claims 1-3, wherein the xyloglucan endotransglycosylase is present at a concentration of about 0.1 nM to about 1 mM.

5. The process of claim 1, wherein the polymeric xyloglucan or polymeric xyloglucan functionalized with a chemical group is present at about 1 mg per g of the material to about 1 g per g of the filler material.

6. The process of claim 1, wherein the xyloglucan oligomer or the functionalized xyloglucan oligomer is present with the polymeric xyloglucan at about 50:1 molar ratio to about 0.5:1 xyloglucan oligomer or functionalized xyloglucan oligomer to polymeric xyloglucan.

7. The process of claim 1, wherein the concentration of polymeric xyloglucan functionalized with a chemical group, polymeric xyloglucan, functionalized xyloglucan oligomer comprising a chemical group, or xyloglucan oligomer incorporated into the material is about 0.01 g to about 500 mg per g of material.

8. The process of claim 1, wherein the xyloglucan oligomer or the functionalized xyloglucan oligomer is present without polymeric xyloglucan or polymeric xyloglucan functionalized with a chemical group at about 1 mg per g of the material to about 1 g per g of the materia.

9. The method of claim 1, wherein the chemical group is a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

10. The process of claim 1, wherein the filler material is selected from the group consisting of alumina, calcium carbonate, calcium sulfate, calcium silicate, glass, kaolin clay, magnesium silicate, mica, red iron oxide, silicon dioxide, titanium dioxide, and combinations thereof.

11. The process of claim 1, wherein the improved property is one or more properties selected from the group consisting of an increase in dry paper strength, an increase in paper density, a decrease in paper sheet thickness, a modification of paper stiffness, an increase in tear strength, improved opacity, improved printability, water resistance, weather resistance, UV or sunlight resistance, resistance to insects or biological pests, antibacterial, antifungal, herbicidal, antiviral, chemical resistance, increased affinity or increased reactivity or increased resistance to compounds of interest, and reduced dusting/linting for a paper, cardboard, or board.

12. The process of claim 1, wherein the improved property can be one or more properties selected from the group consisting of improved paint or coating thickness, fluidity, adhesion to surface, resistance to flaking, cracking or peeling, enhanced strength and durability, improved color or appearance, improved resistance to color fading, improved resistance to sun damage, improved package stability, improved application characteristics, corrosion resistance for paints, coating, sealant, or finish.

13. The process of claim 1, wherein the improved property can be one or more properties selected from the group consisting of an increase in tensile strength, flexibility, resistance to cracking, antimicrobial, antibacterial, antifungal, antiviral, anti-UV or UV-resistant, reduced comedogenic properties, improved optical properties, improved color, improved opacity, improved appearance, fluidity, improved texture, improved compressibility, enhanced stability, resistance to phase-separation, improved viscosity, improved adhesion, and reduced skin-sensitivity for a beauty product or grooming product.

14. The process of claim 1, wherein the improved property can be one or more properties selected from the group consisting of an increase in tensile strength, enhanced mechanical properties, enhanced physical properties, enhanced flexibility or rigidity or brittleness, enhanced UV-protection, enhanced color, enhanced opacity, enhanced resistance to color fading, resistance to flame or flame-retardance, resistance to chemicals, pest resistance, anti-microbial, anti-bacterial, anti-fungal, antiviral, crack resistance, resistance to phase separation, water resistance, reduced weight, enhanced strength per weight, and improved blend ratios of composite materials for a building material.

15. The process of claim 1, wherein the xyloglucan endotransglycosylase is obtainable from a plant or microorganism.

16. The process of claim 15, wherein the plant is selected from the group consisting of a dicotyledon and a monocotyledon.

17. The process of claim 16, wherein the dicotyledon is selected from the group consisting of azuki beans, cauliflowers, cotton, poplar or hybrid aspen, potatoes, rapes, soy beans, sunflowers, thalecress, tobacco, and tomatoes.

18. The process of claim 16, wherein the monocotyledon is selected from the group consisting of wheat, rice, corn and sugar cane.

19. The process of claim 1, wherein the xyloglucan endotransglycosylase is produced by aerobic cultivation of a transformed host organism containing the appropriate genetic information from a plant.

20. A modified filler material made by the process of claim 1.

21. A modified filler comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

22. A suspension comprising a filler at least partly coated with a composition comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group, and a xyloglucan oligomer; (d) a polymeric xyloglucan, and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

23. A process of producing a paper, cardboard, or board, comprising adding the suspension of claim 22 to a fibrous slurry stock in the production of the paper, cardboard, or board.

24. A process of producing a paint, coating, lacquer, or varnish, comprising adding the suspension of claim 22 to a paint stock, a coating stock, a lacquer stock, or a varnish stock in the production of the paint, coating, lacquer, or varnish.

25. A paper comprising the modified filler of claim 21.

26. A cardboard comprising the modified filler of claim 21.

27. A board comprising the modified filler of claim 21.

28. A paint comprising the modified filler of claim 21.

29. A coating comprising the modified filler of claim 21.

30. A beauty or grooming product comprising the modified filler of claim 21.

31. A building material comprising the modified filler of claim 21.

32. A composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer; and (i) a composition of (a), (b), (c), (d), (e), (f), (g), or (h) without a xyloglucan endotransglycosylase.
